(19) United States
(12) Patent Application Publication
(10) Pub. No.: US 2025/0216333 A1
(43) Pub. Date: Jul. 3, 2025

(54) METHODS AND SYSTEMS FOR DETERMINING SURFACE-ENHANCED RAMAN SCATTERING-ACTIVE HOTSPOTS WITH NEAR-FIELD SCANNING OPTICAL MICROSCOPY

(71) Applicant: KING FAHD UNIVERSITY OF PETROLEUM AND MINERALS, Dhahran (SA)

(72) Inventor: Mohammad Kamal HOSSAIN, Dhahran (SA)

(73) Assignee: KING FAHD UNIVERSITY OF PETROLEUM AND MINERALS, Dhahran (SA)

(21) Appl. No.: 18/397,598

(22) Filed: Dec. 27, 2023

(51) Int. Cl. G01N 21/65; C03C 17/36; C03C 17/38

(52) U.S. Cl. CPC G01N 21/658; C03C 17/3649; C03C 17/38; C03C 2218/11

(57) ABSTRACT

A method and system for functionalizing substrates for use in near field surface-enhanced Raman scattering (SERS) spectroscopy. Each functionalized glass substrate of a set of glass substrates functionalized with trimethoxy-[3-(methylamino)propyl] silane is coated with a colloidal solution of gold nanoparticles. A Raman-active dye is applied to the glass substrate through spin coating. A near field SERS spectroscopy of each functionalized glass substrate is performed. Hotspots that produce high-intensity scattering from the dyed immobilized gold nanoparticles are identified for each functionalized glass substrate. A direction of interparticle axis between two adjacent dyed immobilized gold nanoparticles and electromagnetic near field intensity of the scattering along the direction of the interparticle axis for each hotspot are identified for each functionalized glass substrate. A location of each interstitial position, the direction of the interparticle axis, and the electromagnetic near field intensity of the respective hotspot are mapped for each functionalized glass substrate.

### BACKGROUND

### Technical Field

The present disclosure is directed to near-field scanning optical microscopy. In particular, the disclosure is directed to a method for determining surface-enhanced Raman scattering-active hotspots with near-field scanning optical microscopy.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Raman imaging is a non-destructive analytical technique used in microscopy and spectroscopy to visualize the chemical composition and distribution of substances within a sample. It is based on the Raman scattering phenomenon, which occurs when monochromatic light (for example, laser light) interacts with molecules, leading to a shift in wavelength due to molecular vibrations. In Raman imaging, a sample is illuminated with a laser, and the scattered light is collected and analyzed. The resulting Raman spectrum provides information about the molecular vibrations within the sample, offering insights into its chemical composition. By scanning the laser across the sample and recording the Raman spectra at different points, a spatial map of the chemical composition of the sample can be generated. Raman imaging is particularly valuable for studying biological samples, polymers and pharmaceuticals, and may be a valuable technique used in materials science. It allows researchers to visualize the distribution of specific molecules, identify different phases in a material, and study changes in molecular structures. In imaging, nano sized silver/gold colloids and roughened metallic substrates are used to amplify the intensity of the Raman scattering of adsorbed molecules via surface-enhanced Raman scattering (SERS). This can increase the sensitivity and/or the specificity of the analysis.

SERS is a highly sensitive analytical technique that combines Raman spectroscopy with the enhancement of signals provided by the presence of nanostructured metallic surfaces. SERS is used to amplify the Raman signals of molecules adsorbed on or near these surfaces, enabling the detection of trace amounts of substances. In SERS, a sample is placed on or near a substrate that has nanostructures made of metals such as gold or silver. When the sample is illuminated with a laser, the Raman signals from the molecules are significantly enhanced due to the localized surface plasmon resonance of the metallic nanostructures. The enhancement can be several orders of magnitude greater than what is observed in conventional Raman spectroscopy. SERS is known for its high sensitivity, allowing the detection of molecules at extremely low concentrations. Two mechanisms are known for the enhancement in SERS, one is the electromagnetic (EM) enhancement due to localized EM field distribution at the “hotspots”, and the second is the chemical enhancement due to charge transfer between the target analyte and a SERS-active electrode. In the SERS enhancement, the EM enhancement effect through the “hotspot” mechanism is recognized to be several orders higher than that of the chemical enhancement. “Hotspot” or “active site” is defined as the site of interest where a significant and localized electromagnetic (EM) field exists and thus contributes to SERS enhancement. The emission of an individual molecule at SM-SERS conditions may depend on the local enhancement field of hotspots, as well as the binding affinity and positioning at a hot spot region. In this regard, the stability of near-field nano-optics at hotspots is critical, particularly in a biological milieu. SERS is based on an understanding of the Raman phenomenon. By definition, it is an inelastic light scattering technique, where incident photons interact with an analyte via the polarizability tensor of the system, either depositing a quantum of energy into an excitation state (Stokes scattering) or acquiring a quantum of energy from an excitation state (anti-Stokes scattering). Beyond the particular polarization matrix elements, the Raman scattering rate is proportional to the electromagnetic intensity at the molecule and to the density of states available for the outgoing Raman-scattered photon. Through a combination of geometry and metal dielectric function, metal nanostructures have a frequency-dependent optical response, with contributions from plasmons. The plasmon response involves the displacement of charge density from its equilibrium position on a scale of length generally smaller than the excitation wavelength. As a result, local areas of high electromagnetic field increase and plasmonic hot spots are formed for Raman scattering enhancement.

Due to the presence of robust electromagnetic fields, gap-mode plasmonics are confined within a nanometric hotspot volume. In this context, even a minute alteration in the nanometric geometry may establish new pathways for the contained energy to propagate through the coupling of adjacent hotspots, thereby altering the distributions of the electromagnetic near-field. However, there are consistent challenges in comprehending the microscopic correlation between localized electromagnetic near-field and confined optical fields. The size of the hotspot, typically ranging from 1 to 10 nm, falls well below the optical diffraction limit, posing a challenging task in characterizing hotspots experimentally. Consequently, the resolution of the SERS measurements linked to electromagnetic (EM) hotspots is limited to the 200-400 nm length scale, which is significantly larger than the actual relevant length scale of the hotspot. An obstacle in capturing SERS-active hotspots lies in achieving reproducibility in SERS substrate fabrication. Precise control over the nanoscale features, such as size, shape, and distribution, crucial in achieving SERS-active hotspots, is challenging with conventional fabrication methods. This can lead to variability in SERS response, impeding the reliable capture of hotspots required for high-sensitivity detection.

In the realm of optical microscopy, the nanoscale imaging of optical confinement has long been a sought-after goal and presents a persistent challenge. Spectrally-resolved and spatially-resolved measurements, especially correlated observations of such hotspots, have been infrequently reported and remain challenging due to the diffraction limit, a fundamental constraint on the maximum resolution of an optical image. The diffraction theory of light calculates the possible spatial resolution of the collected image, indicating a maximum resolution of hundreds of nanometers in the visible and up to several microns in the infrared. However, for understanding and interpreting near-field optics governed by nanoplasmonics and nanophononics, this resolution is inadequate. To overcome the resolution challenge, near-field scanning optical microscopy (NSOM) was developed. NSOM enables spectrally and spatially resolved measurements by collecting or facilitating the extraction of emitted or scattered photons. Near-field tips, or NSOM probes, primarily modify or enhance the incident optical field locally within the nanometric geometry. This has a significant impact on any surface-sensitive optical process, such as SERS. Snapshots of hotspots have been obtained by NSOM with a resolution of about 10 nm, and scanning (transmission) electron microscopes have revealed images with a resolution of more than 10 nm utilizing electron energy-loss spectroscopy. However, most single-molecule SERS studies have focused on the performance of SERS-active substrates, avoiding qualitative and quantitative measures of SERS hotspots due to experimental setup limitations.

Accordingly, it is one object of the present disclosure to provide methods and systems for characterizing gold nanoparticle surface-enhanced Raman scattering-active hotspots with near-field scanning optical microscopy.

### SUMMARY

In an exemplary embodiment, a method for functionalizing substrates for use in near field surface-enhanced Raman scattering (SERS) spectroscopy is described. The method includes coating each functionalized glass substrate of a set of glass substrates functionalized with trimethoxy-[3-(methylamino)propyl] silane with a colloidal solution of gold nanoparticles suspended in water. The trimethoxy-[3-(methylamino)propyl] silane immobilizes the gold nanoparticles on each functionalized glass substrate. The method also includes applying a Raman-active dye to the glass substrate having immobilized gold nanoparticles. In addition, the method includes performing near field SERS spectroscopy of each functionalized glass substrate coated with dyed immobilized gold nanoparticles and identifying, from the near field SERS spectroscopy, for each functionalized glass substrate coated with dyed immobilized gold nanoparticles, hotspots which produce high intensity scattering from the dyed immobilized gold nanoparticles, wherein each hotspot is located at an interstitial position between two adjacent dyed immobilized gold nanoparticles.

The method includes identifying for each functionalized glass substrate coated with dyed immobilized gold nanoparticles, a direction of an interparticle axis between the two adjacent dyed immobilized gold nanoparticles and an electromagnetic near field intensity of the high intensity scattering along the direction of the interparticle axis for each hotspot. The method also includes mapping, for each functionalized glass substrate coated with dyed immobilized gold nanoparticles, by a computing device, a location of each interstitial position of each hotspot, the direction of the interparticle axis of the respective hotspot, and the electromagnetic near field intensity of the respective hotspot. The method further includes assigning a substrate identification number to each glass substrate coated with dyed immobilized gold nanoparticles, and storing, in a database, the location of each interstitial position of each hotspot, the direction of the interparticle axis of the respective hotspot, and the electromagnetic near field intensity of the respective hotspot with the substrate identification number.

In another exemplary embodiment, a method of identifying a target analyte is described. The method includes obtaining a functionalized glass substrate coated with dyed immobilized gold nanoparticles and coating the functionalized glass substrate coated with dyed immobilized gold nanoparticles with a target analyte. The functionalized glass substrate coated with dyed immobilized gold nanoparticles has known positions of high intensity scattering from interparticle axes between adjacent dyed immobilized gold nanoparticles. In addition, the method includes performing near field SERS spectroscopy at the known positions by directing a laser beam having a p-polarization along a direction parallel to a direction of the interparticle axis of each known position, receiving, by a computing device, SERS spectra for each of the known positions, comparing the SERS spectra for each of the known positions to a database record of known SERS spectra of molecules and identifying molecules in the target analyte based on matching the SERS spectra to the database record of known SERS spectra of molecules.

In another exemplary embodiment, a system for functionalizing glass substrates for use in detecting target molecules with near field surface-enhanced Raman scattering (SERS) spectroscopy is described. The system includes a glass substrate coated with immobilized gold nanoparticles. Each glass substrate has an identification number. The system includes a coating of Raman-active dye configured to adhere to the molecule in the target analyte. The coating of Raman-active dye is applied by spin coating. In addition, the system includes an aperture near-field scanning optical microscope (a-NSOM) configured to perform near field SERS spectroscopy of each functionalized glass substrate coated with the dyed immobilized gold nanoparticles, and a computing device connected to the a-NSOM. The computing device includes electric circuitry, a memory configured to store program instructions and at least on processor configured to execute the program instructions to identify, for each functionalized glass substrate coated with the dyed immobilized gold nanoparticles, positions of hotspots which produce high intensity scattering from the dyed immobilized gold nanoparticles. Each hotspot is located at an interstitial position between two adjacent dyed immobilized gold nanoparticles. The a-NSOM is further configured to perform sheer force measurements simultaneously with the near-field SERS spectroscopy and generate a contour map configured to show the interparticle axes of each hotspot. The a-NSOM is further configured to measure an electromagnetic near field intensity along the interparticle axes of each hotspot. The computing device is further configured to identify a direction of the interparticle axis between the two adjacent dyed immobilized gold nanoparticles of each hotspot based on the contour map. The system also includes a database connected to the computing device. The database is configured to store the identification number of each glass substrate, the position of each hotspot, the direction of the interparticle axis between the two adjacent dyed immobilized gold nanoparticles of each hotspot, the electromagnetic near field intensity along the interparticle axes of each hotspot and the contour map.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is a schematic diagram of a near-field scanning optical microscope (NSOM) device setup.

FIG. 2 is a process flow of a method for functionalizing substrates for use in a near field surface-enhanced Raman scattering (SERS) spectroscopy, according to certain embodiments.

FIG. 3 is a process flow for a method of identifying a target analyte, according to certain embodiments.

FIG. 4A shows a shear-force topography of an Au nanoaggregate captured simultaneously during near-field SERS measurements using a-NSOM, according to certain embodiments.

FIG. 4B represents near-field SERS spectra of R6G recorded at four specific sites of interest, according to certain embodiments.

FIG. 4C shows a near-field SERS mapping obtained at a near-field SERS band of 1647 cm⁻¹ of R6G, according to certain embodiments.

FIG. 4D shows a histogram of the displacement versus the detected event count, according to certain embodiments.

FIG. 5A shows a near-field SERS mapping overlapped with possible constituent nanoparticles, according to certain embodiments.

FIG. 5B shows near-field SERS spectra of R6G recorded at five sites of interest, according to certain embodiments.

FIG. 5C shows a high-level view of near-field SERS mapping of R6G recorded at five sites of interest, according to certain embodiments.

FIG. 5D shows a contour plot of the near-field SERS intensity overlapped with near-field SERS mapping, according to certain embodiments.

FIG. 6A shows a near-field SERS mapping of R6G extracted for the 1647 wavenumber/cm band, according to certain embodiments.

FIG. 6B shows a near-field SERS mapping of R6G extracted for the 1572 wavenumber/cm band, according to certain embodiments.

FIG. 6C shows a near-field SERS mapping of R6G extracted for the 1329 wavenumber/cm band, according to certain embodiments.

FIG. 6D shows a near-field SERS mapping of R6G extracted for the 1185 wavenumber/cm band, according to certain embodiments.

FIG. 6E shows a near-field SERS mapping of R6G extracted for the 1238 wavenumber/cm band, according to certain embodiments.

FIG. 6F shows a near-field SERS mapping of R6G extracted for the 1078 wavenumber/cm band, according to certain embodiments.

FIG. 6G shows a near-field SERS mapping of R6G extracted for the 887 wavenumber/cm band, according to certain embodiments.

FIG. 6H shows a near-field SERS mapping of R6G extracted for the 748 wavenumber/cm band, according to certain embodiments.

FIG. 6I shows shear-force topography of a given area captured simultaneously during near-field SERS measurements, according to certain embodiments.

FIG. 7A shows a near-field SERS mapping of R6G extracted for the 1746 wavenumber/cm band, according to certain embodiments.

FIG. 7B shows a near-field SERS mapping of R6G extracted for the 1622 wavenumber/cm band, according to certain embodiments.

FIG. 7C shows a near-field SERS mapping of R6G extracted for the 1458 wavenumber/cm band, according to certain embodiments.

FIG. 7D shows a near-field SERS mapping of R6G extracted for the 1422 wavenumber/cm band, according to certain embodiments.

FIG. 7E shows a near-field SERS mapping of R6G extracted for the 832 wavenumber/cm band, according to certain embodiments.

FIG. 7F shows a near-field SERS mapping of R6G extracted for the 649 wavenumber/cm band, according to certain embodiments.

FIG. 8A represents EM near-field distributions at XY (Z=0) plane for a defined model geometry excited with incident excitation of s-polarization, according to certain embodiments.

FIG. 8B shows EM near-field distributions along the XY (Z=0) plane of the same model excited with p-polarization of incident excitation, according to certain embodiments.

FIG. 8C displays EM near-field distributions along the XY (Z=0) plane of the same model excited with incident excitation of oblique (45°)-polarization.

FIG. 9 is an illustration of a non-limiting example of details of computing hardware used in the computing system, according to certain embodiments.

FIG. 10 is an exemplary schematic diagram of a data processing system used within the computing device, according to certain embodiments.

FIG. 11 is an exemplary schematic diagram of a processor used with the computing device, according to certain embodiments.

FIG. 12 is an illustration of a non-limiting example of distributed components which may share processing with the controller of the computing device, according to certain embodiments.

### DETAILED DESCRIPTION

In the drawings, like reference numerals designate identical or corresponding parts throughout the several views. Further, as used herein, the words “a”, “an” and the like generally carry a meaning of “one or more”, unless stated otherwise.

Furthermore, the terms “approximately,” “approximate”, “about” and similar terms generally refer to ranges that include the identified value within a margin of 20%, 10%, or preferably 5%, and any values therebetween.

Aspects of this disclosure are directed to a method, device, and system for determining surface-enhanced Raman scattering-active hotspots with near-field scanning optical microscopy.

Raman spectroscopy using surface enhanced Raman scattering (SERS) nanoparticles is an exciting new molecular imaging modality that has emerged over the past decade. The approach is based on the inelastic scatter of photons by molecular bonds. Small-molecule dyes adsorbed on a rough metal (usually gold) surface experience a dramatic increase in their Raman scatter intensity due to surface plasmons, which induce strong localized electric fields near the surface. As each bond within a Raman dye has a characteristic vibrational energy, the SERS spectrum is unique for that dye. Determining surface-enhanced Raman scattering-active hotspots with near-field scanning optical microscopy using SERS is described.

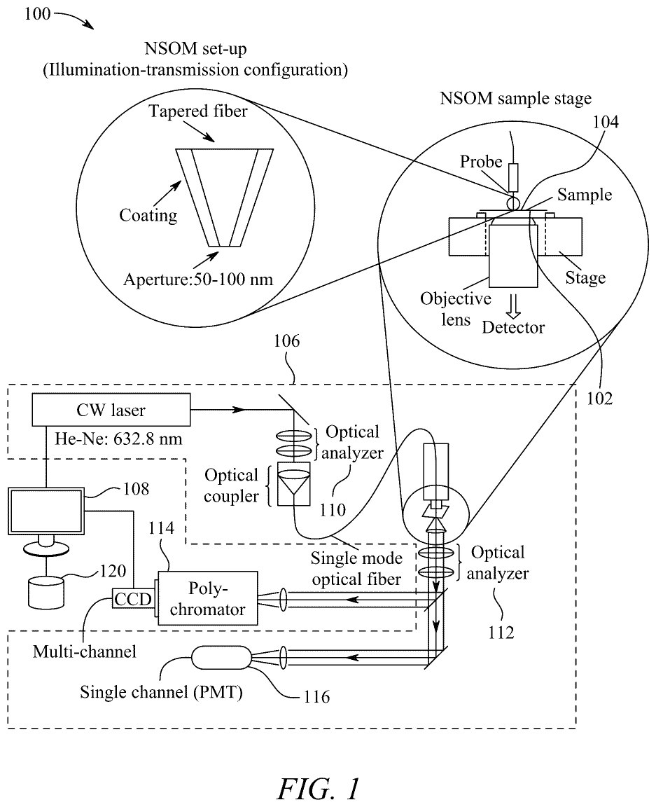

FIG. 1 is a schematic diagram of a system 100 depicting an aperture near-field scanning optical microscope (a-NSOM) setup for use in detecting target molecules with near field SERS spectroscopy. The system 100 includes a glass substrate 102, a coating of Raman-active dye 104, an aperture near-field scanning optical microscope (a-NSOM) 106, and a computing device 108.

The glass substrate 102 is a flat, rigid material made of glass that serves as a base or support for various applications, such as electronic devices, optics, and coatings. The glass substrate 102 is used herein for optics on which immobilized gold nanoparticles are coated. There may be a plurality of glass substrates 102, and each glass substrate may have a unique identification number. A coating of Raman-active dye 104 may refer to an application of a layer or film of a Raman-active substance onto a substrate or surface (such as the glass substrate). In the context of Raman spectroscopy, a Raman-active dye is a molecule that exhibits Raman scattering, meaning it can produce intense and characteristic Raman signals when illuminated with laser light. In other words, the coating enhances Raman signals through the SERS effect. Examples of Raman-active dyes include rhodamine 6G, crystal violet, methylene blue, malachite green, nile red, congo red, methyl orange, and acridine orange. SERS involves the use of nanostructured surfaces coated with Raman-active molecules to significantly amplify Raman signals, allowing for highly sensitive detection. In the present disclosure, the coating of Raman-active dye is configured to adhere to the molecule in the target analyte. In an example, the coating of Raman-active dye is applied by spin coating. Other examples of coating not described herein are contemplated. For example, the coating of Raman-active dye may be applied through dip coating or spray coating.

The a-NSOM 106 is a type of near-field scanning optical microscope (NSOM) that utilizes an aperture or a small opening at the end of a sharp metallic tip to achieve nanoscale optical resolution. The a-NSOM 106 are designed to overcome the diffraction limit of traditional optical microscopes, allowing for imaging and spectroscopy at a spatial scale much smaller than the wavelength of light. In the a-NSOM 106, an aperture at a tip of a metallic probe is defined smaller than a wavelength of light, allowing for subwavelength spatial resolution. The aperture enables near-field interactions between the sample and the tip, allowing for the collection and localization of light signals at a nanoscale. This technique helps in studying optical properties and interactions with nanoscale features on a sample. The a-NSOM 106 is configured to perform near field SERS spectroscopy of each functionalized glass substrate coated with the dyed immobilized gold nanoparticles. The a-NSOM 106 includes a source, an optical analyzer 110, an optical coupler, a metallic probe having a tapered fiber with an aperture of 50-100 nm, an optical analyzer 112, a polychromator-CCD (for multichannel detection) and/or an avalanche photodiode 116 (for single-channel detection). In operation, the source, which can be a continuous-wave laser, emits a continuous and steady beam of light. The light is processed through the optical analyzer 110 and the optical coupler to be delivered at the glass substrate 102 through the metallic probe via a tapered fiber. The incidence of light on the glass substrate 102 causes a Raman photon scattering. The scattered Raman photon is collected by an objective lens mounted below the sample stage and detected by a polychromator-CCD (for multichannel detection) and/or an avalanche photodiode 116 (for single-channel detection). The a-NSOM 106 is further configured to perform sheer force measurements simultaneously with the near-field SERS spectroscopy and generate a contour map configured to show the interparticle axes of each hotspot. The hotspot are located at an interstitial position (for example, spaces or gaps) between two adjacent dyed immobilized gold nanoparticles. The a-NSOM 106 is further configured to measure an electromagnetic near field intensity along interparticle axes of each hotspot.

The computing device 108 connected to the a-NSOM 106 is configured to execute the program instructions to identify, for each functionalized glass substrate 102 coated with the dyed immobilized gold nanoparticles, positions of hotspots that produce high intensity scattering from the dyed immobilized gold nanoparticles. The computing device 108 is also configured to identify a direction of the interparticle axis between the two adjacent dyed immobilized gold nanoparticles of each hotspot based on the contour map.

The computing device 108 is connected to a database 120. The database 120 is configured to store the identification number of each glass substrate, the position of each hotspot, the direction of the interparticle axis between the two adjacent dyed immobilized gold nanoparticles of each hotspot, the electromagnetic near field intensity along the interparticle axes of each hotspot and the contour map.

In operation, the a-NSOM 106 was initiated by exciting an excitation source which is a continuous wave laser (for example, He—Ne laser of 632.8 nm). The laser is directed at samples placed on the functionalized glass substrate 102 through a single-mode optical fiber through the optical coupler. The other end of the optical fiber was used as an a-NSOM 106 probe. A gold-coated apertured near-field probe tip (aperture diameter 50-100 nm) was used to localize the excitation, and the measurements were carried out in an illumination-transmission configuration under ambient conditions. The tip-to-specimen distance was kept as low, for example, to 10-15 nm by the tuning-fork feedback mechanism. Morphology of the sample surface was captured by shear-force topographic measurements captured simultaneously by the a-NSOM 106. The shear-force topography measurement involved the use of a sharp probe to characterize the topography of a sample surface. The scattered Raman photon was collected by an objective lens mounted below the sample stage and detected by a polychromator-CCD 114 (for multichannel detection) and/or an avalanche photodiode 116 (for single-channel detection).

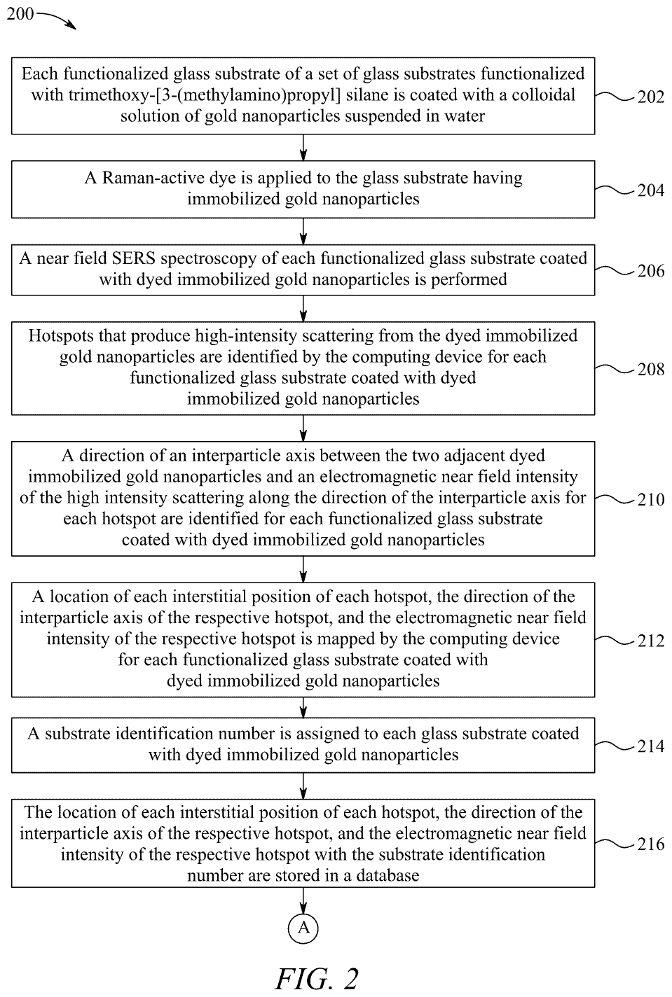

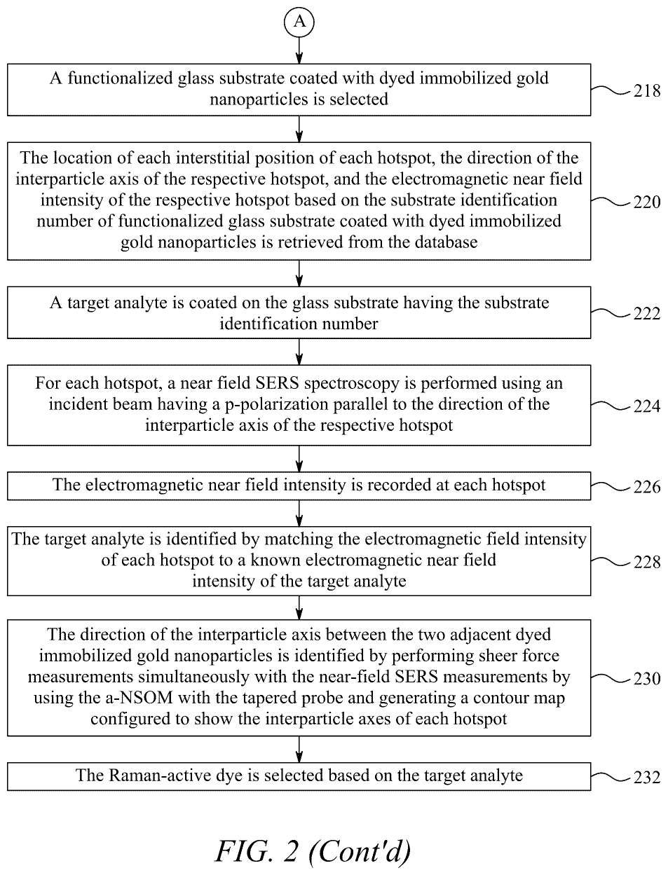

FIG. 2 is a process flow of a method for functionalizing substrates for use in near field SERS spectroscopy. In step 202, each functionalized glass substrate 102 of a set of glass substrates 102 functionalized with trimethoxy-[3-(methylamino)propyl] silane is coated with a colloidal solution of gold nanoparticles suspended in water. The trimethoxy-[3-(methylamino)propyl] silane immobilizes the gold nanoparticles on each functionalized glass substrate 102. The gold nanoparticles may each have a diameter in a range of 96.0 nm to 104.0 nm.

In step 204, a Raman-active dye 104 is applied to the glass substrate 102 having immobilized gold nanoparticles. In implementations, the Raman-active dye 104 is applied onto each functionalized glass substrate 102 coated with the immobilized gold nanoparticles by spin coating. The spin coating distributes the Raman-active dye 104 across each functionalized glass substrate 102 coated with the immobilized gold nanoparticles.

In step 206, a near field SERS spectroscopy of each functionalized glass substrate 102 coated with dyed immobilized gold nanoparticles is performed.

In step 208, hotspots that produce high-intensity scattering from the dyed immobilized gold nanoparticles are identified by the computing device 108 for each functionalized glass substrate 102 coated with dyed immobilized gold nanoparticles. Each hotspot is located at an interstitial position between two adjacent dyed immobilized gold nanoparticles.

In step 210, a direction of an interparticle axis between the two adjacent dyed immobilized gold nanoparticles and an electromagnetic near field intensity of the high intensity scattering along the direction of the interparticle axis for each hotspot are identified for each functionalized glass substrate 102 coated with dyed immobilized gold nanoparticles.

In step 212, a location of each interstitial position of each hotspot, the direction of the interparticle axis of the respective hotspot, and the electromagnetic near field intensity of the respective hotspot is mapped by the computing device 108 for each functionalized glass substrate 102 coated with dyed immobilized gold nanoparticles.

In step 214, a substrate identification number is assigned to each glass substrate 102 coated with dyed immobilized gold nanoparticles; and

In step 214, the location of each interstitial position of each hotspot, the direction of the interparticle axis of the respective hotspot, and the electromagnetic near field intensity of the respective hotspot with the substrate identification number are stored in a database 120.

In step 216, a functionalized glass substrate 102 coated with dyed immobilized gold nanoparticles is selected.

In step 218, the location of each interstitial position of each hotspot, the direction of the interparticle axis of the respective hotspot, and the electromagnetic near field intensity of the respective hotspot based on the substrate identification number of functionalized glass substrate 102 coated with dyed immobilized gold nanoparticles is retrieved from the database 120.

In step 220, a target analyte is coated on the glass substrate 102 having the substrate identification number.

In step 222, for each hotspot, a near field SERS spectroscopy is performed using an incident beam having a p-polarization parallel to the direction of the interparticle axis of the respective hotspot. In implementation, the near field SERS spectroscopy is performed using the a-NSOM 106. Performing near field SERS spectroscopy with the a-NSOM 106 includes equipping the a-NSOM 106 with a tapered probe and emitting the incident beam having a p-polarization from a tip of the tapered probe. In implementations, a gold coating is deposited on the tip of the tapered probe.

In step 224, the electromagnetic near field intensity is recorded at each hotspot.

In step 226, the target analyte is identified by matching the electromagnetic field intensity of each hotspot to a known electromagnetic near field intensity of the target analyte.

In step 228, the direction of the interparticle axis between the two adjacent dyed immobilized gold nanoparticles is identified by performing sheer force measurements simultaneously with the near-field SERS measurements by using the a-NSOM 106 with the tapered probe and generating a contour map configured to show the interparticle axes of each hotspot.

In step 230, the Raman-active dye 104 is selected based on the target analyte.

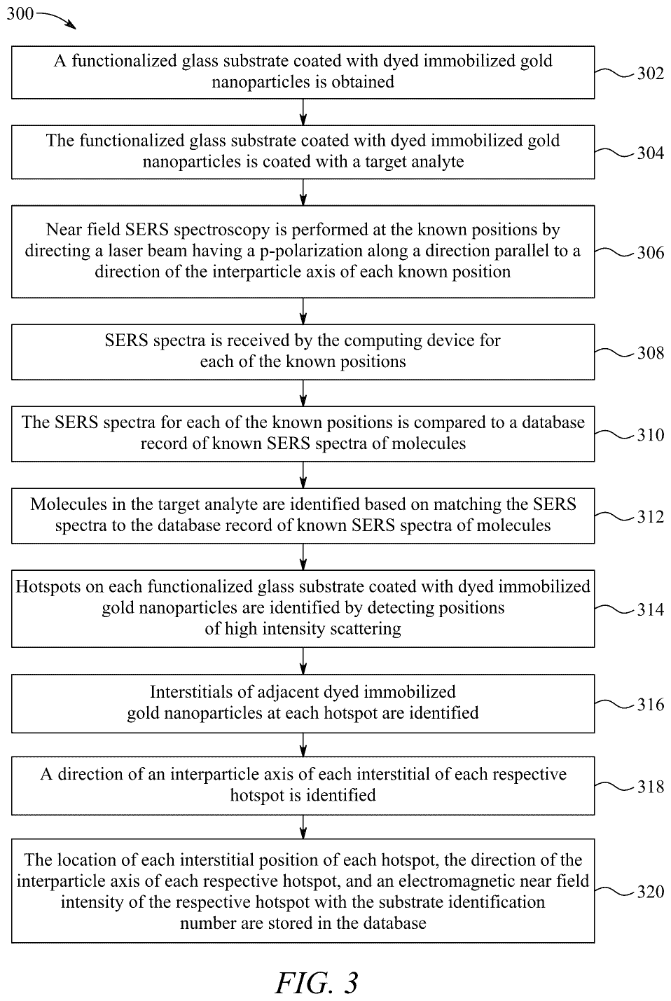

FIG. 3 is a process flow for a method of identifying a target analyte. In step 302, a functionalized glass substrate 102 coated with dyed immobilized gold nanoparticles is obtained. In an example, the dye is a Raman-active dye 104. In an example, the Raman-active dye 104 is Rhodamine 6G. In examples, the gold nanoparticles each have a diameter in a range of 96.0 nm to 104.0 nm.

In step 304, the functionalized glass substrate 102 coated with dyed immobilized gold nanoparticles is coated with a target analyte. The functionalized glass substrate 102 coated with dyed immobilized gold nanoparticles has known positions of high intensity scattering from interparticle axes between adjacent dyed immobilized gold nanoparticles.

In step 306, near field SERS spectroscopy is performed at the known positions by directing a laser beam having a p-polarization along a direction parallel to a direction of the interparticle axis of each known position. The near field SERS spectroscopy is performed by directing a tip of a tapered probe of an aperture near-field scanning optical microscope (a-NSOM 106) in the direction of the interparticle axis of each known position. The tip of the tapered probe is coated with gold.

In step 308, SERS spectra is received by the computing device 108 for each of the known positions. In step 310, the SERS spectra for each of the known positions is compared to a database 120 record of known SERS spectra of molecules.

In step 312, molecules in the target analyte are identified based on matching the SERS spectra to the database 120 record of known SERS spectra of molecules.

In implementations, each functionalized glass substrate 102 coated with dyed immobilized gold nanoparticles is tagged with a substrate identification number.

In step 314, hotspots on each functionalized glass substrate 102 coated with dyed immobilized gold nanoparticles are identified by detecting positions of high intensity scattering; In step 316, identifying interstitials of adjacent dyed immobilized gold nanoparticles at each hotspot.

In step 318, a direction of an interparticle axis of each interstitial of each respective hotspot is identified;

In step 320, the location of each interstitial position of each hotspot, the direction of the interparticle axis of each respective hotspot, and an electromagnetic near field intensity of the respective hotspot with the substrate identification number are stored in the database 120.

In aspects, identifying the direction of the interparticle axis between the two adjacent dyed immobilized gold nanoparticles at each hotspot includes performing sheer force measurements simultaneously during the near-field SERS measurements using the a-NSOM 106 with the tapered probe, generating a contour map based on the sheer force measurements, determining the hotspots from the positions of high intensity scattering observed from the near-field SERS measurements, determining the interparticle axes of each hotspot from the contour map, and determining a direction of the interparticle axis of each hotspot by observing the strength of the high intensity scattering along each interparticle axis.

The glass substrate 102 having immobilized gold nanoparticles is coated with a dye by selecting a dye based on the target analyte, obtaining the selected dye, applying a drop of the selected dye to the glass substrate 102 having immobilized gold nanoparticles, inserting the functionalized glass substrate 102 coated with the immobilized gold nanoparticles into a spin coating machine, spin coating the dye onto each functionalized glass substrate 102 coated with the immobilized gold nanoparticles. The spin coating distributes the dye across each functionalized glass substrate 102 coated with the immobilized gold nanoparticles.

Experiments and analysis are described below. To validate hotspot mechanisms in SERS, a monolayer and well-defined aggregate was achieved. To achieve the monolayer and well-defined aggregate, a high-purity colloidal solution of gold nanoparticles was suspended in water. In examples, the gold nanoparticles had various dimensions with mean diameter being 98.9 nm, a mean range of 96.0 to 104.0 nm and a concentration of 5.6×109 nanoparticles/mL. In a non-limiting example, the gold nanoparticles were procured from British BioCell International (Berry Smith Llp, Haywood House, Dumfries Place, Cardiff, CF10 3GA). A glass substrate 102 functionalized with trimethoxy-[3-(methylamino)propyl] silane was procured from Matsunami (Matsunami Glass USA Inc. 1971 Midway Ln Ste J Bellingham, WA, 98226-7682 United States) and used as a substrate to immobilize gold nanoparticles atop. A salient feature of N-particle gold nanoaggregates where Nis small in number was confirmed by scanning electron microscope (for example, scanning electron microscope, JEOL FE6500 manufactured by JEOL USA, Inc. 11 Dearborn Road, Peabody, MA 01960) and shear-force topographic measurements of the a-NSOM 106. It was revealed that the constituent nanoparticles were not in direct contact, and therefore the tiny gaps played a crucial role in generating SERS-active hotspots.

Near-field SERS-activity in the presence of a well-defined gold nanoaggregate was carried out using a Raman-active dye 104, Rhodamine 6G (R6G, C28H31N2O3Cl). R6G was used as procured from Chroma GesellschaftSchmid GMBH & Co (Chroma-Gesellschaft Schmid GmbH & Co. KG, Stuttgart, Germany) without any modifications. The gold nanoaggregate immobilized on the coverslip was spin-coated with a water solution of R6G, and thus analytes spread out due to strong centrifugal force. A small droplet of ca. 50-100 nL solution of R6G was used in this case. It was noticed that only around 10% of the molecules contained in the droplet were adsorbed on the substrate. High-speed spin coating with such a small volume of diluted solution does not cause aggregations on the sample. The coverage of R6G molecules was estimated to be several hundred molecules per 100 nm×100 nm considering the homogenous distribution of the solution. Near-field SERS measurements were carried out at ca. 40 nm steps across the scan area of 1 μm×1 μm with a 1 second exposure time. Further, a Finite-Difference Time-Domain (FDTD) calculation was carried out. The FDTD is a numerical method used in computational electromagnetics to analyze and model electromagnetic wave interactions with structures and materials. The FDTD is also used in for modeling nano-scale optical devices.

As shown in FIG. 1, a model system 100 was set up such that the geometry and parameters represented the nanoaggregate under investigation and the NSOM setup closely. A close-packed and interacting tetramer along with two isolated nanoparticles very similar to those observed in morphological investigations was developed and simulated by Planc FDTD. Electromagnetic near-field distributions along various planes for s-, p-, and 45° incident polarizations were extracted and analyzed to correlate with those obtained in near-field SERS observations.

In order to correlate the EM near-field distribution, a 632.8 nm excitation normal to the model geometries was utilized in the FDTD simulation. To analyze in a simplified manner, nano-objects were assumed to be smooth and spherical, despite the fact that the individual nanoparticles differed from one another, notably in size and form, as seen in topographical studies.

SERS detects strong EM near-field distribution (known as the EM enhancement mechanism) induced at the hotspot. Therefore, the characteristics of the hotspot as well as the incident field define whether incident energy is confined at the interstitial region, and thus energizes the analyte positioned at that location. The number of interstitials increases with the number of constituent nanoparticles, where EM near-field distributions become hybridized and merge with nearby distributions.

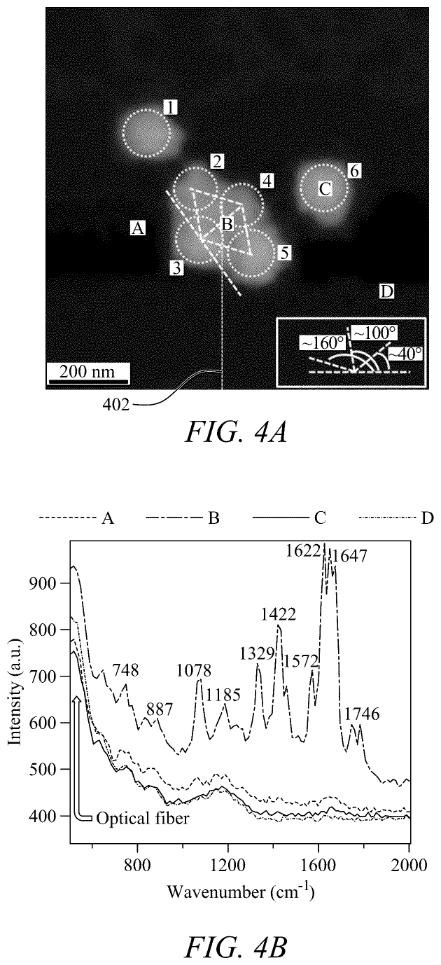

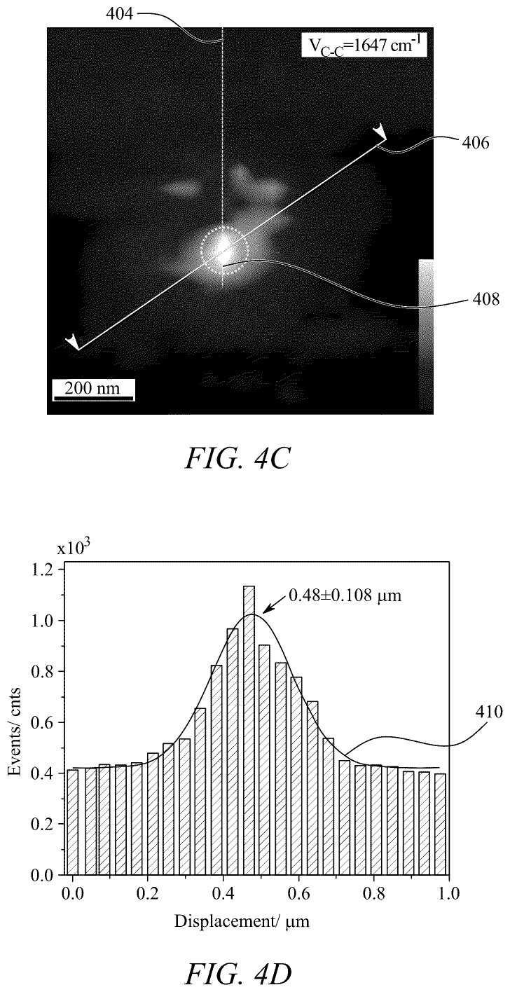

FIG. 4A shows the shear-force topography of an Au nanoaggregate captured simultaneously during the near-field SERS measurements using an a-NSOM 106 facility. An aggregate of six distinct nanoparticles were noted, where four nanoparticles marked as “2”, “3”, “4”, and “5” closely interacted, yielding four interstitials along three different interparticle axes. Nanoparticles marked “1” and “6” were isolated. The directions of the interparticle axis between constituent nanoparticles are shown in the white rectangle of FIG. 4A. The interparticle axes between nanoparticles “2” and “3” and nanoparticles “4” and “5” were estimated to be ˜100°, whereas those between nanoparticles “2” and “4” and nanoparticles “3” and “5” were along ˜160°. The interparticle axis between nanoparticles “3” and “4” was estimated to be ˜40°. It can be seen that the strongest EM near-field distribution is achieved at the perfect match between the interparticle axis and incident polarization due to constructive plasmon coupling. Detailed EM near-field distributions through FDTD simulation are described below. However, the direction of the interparticle axis is considered to elucidate the SERS enhancement at a particular hotspot. A snapshot of such a hotspot through correlated optometrology augments some of the SERS characteristics. As elaborated herein, the entire specimen surface was scanned by the near-field probe of the a-NSOM 106, and the SERS spectra were collected from each pixel of the mapping. FIG. 4B represents near-field SERS spectra of R6G recorded at four specific sites of interest as marked by “A” (nearby the core aggregate), “B” (at the interstitial between nanoparticles “3” and “4”), “C” (at the monomer marked as “6”), and “D” (far from the core aggregate). It was evident that near-field SERS of R6G were strongly enhanced at “B” compared to those observed at “A”, “C” and “D”. A few near-field SERS bands of R6G at 748, 887, 1078, 1187, 1329, 1422, 1572, 1622, 1647, and 1746 cm⁻¹ were observed and labelled in FIG. 4B. The labelled near-field SERS bands coincided well with the noted measurements. Also, there were several unknown or shifted near-field SERS bands of R6G which were recorded during the instance. The detailed near-field SERS bands, band assignments, and the corresponding mappings have been explained below. FIG. 4C shows a near-field SERS mapping obtained at the near-field SERS band of 1647 cm⁻¹ (aromatic C—C stretching mode) of R6G. A vertical white dashed line across FIG. 4A (line 402) and FIG. 4C (line 404) supported the observer in correlating the shear-force topology and the corresponding near-field SERS mapping. Intensity distributions of the mapping revealed that intense near-field SERS was observed at the interstitial of nanoparticles “3” and “4”. The intensities of the near-field SERS band 1647 cm⁻¹ of R6G (aromatic C—C stretching mode) along the line 406 as shown in FIG. 4C were plotted in FIG. 4D along with a Gaussian fit as shown as curve 410. It was noted that a full width at half maximum (FWHM) of the maximum near-field SERS intensity was spread over ±108 nm, as shown in FIG. 4D. As seen in FIG. 4C, there were several sites of intense near-field SERS around the strongest hotspot (marked by 408). The spectral characteristics of those sites were analyzed further and stated below. FIG. 4D shows a histogram of the displacement versus the detected event count.

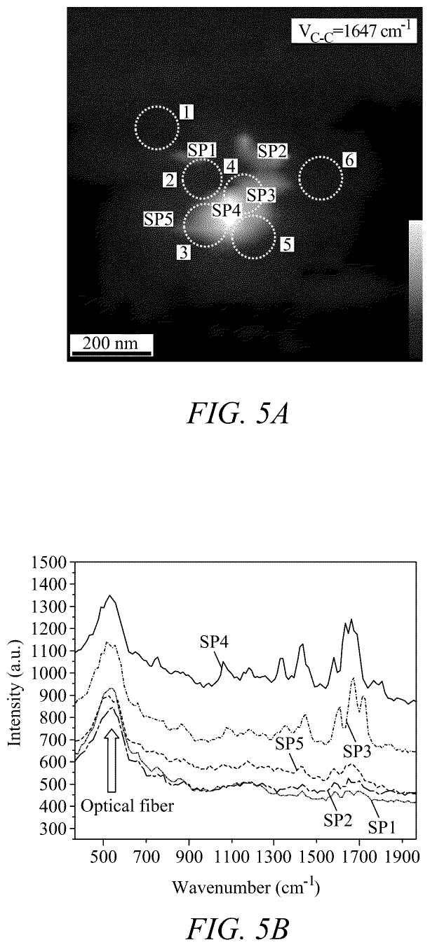

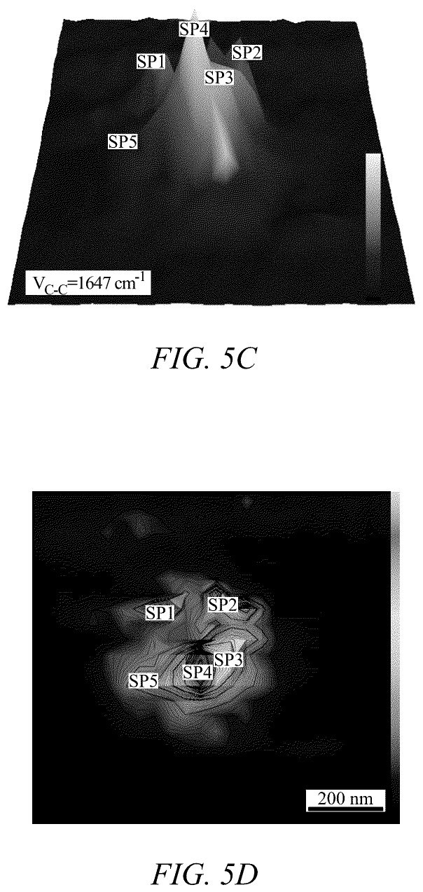

FIG. 5A shows a near-field SERS mapping (1647 cm⁻¹, aromatic C—C stretching mode of R6G) overlapped with possible constitute nanoparticles. From the many intense near-field SERS sites, five sites of interest were depicted as marked by “SP1” (at the top of nanoparticle “2”), “SP2” (in between nanoparticles “4” and “6”), “SP3” (next to nanoparticle “4”), “SP4” (at the interstitial of nanoparticles “4” and “3”) and “SP5” (to the left of nanoparticle “3”) as shown in FIG. 5A. The near-field SERS spectra of R6G recorded at the abovementioned five sites of interest are shown in FIG. 5B. At “SP4”, most of the SERS bands were found strongly enhanced in the near-field SERS spectrum of R6G. Some of the SERS bands were found to coincide well with known measurements, as explained earlier, whereas others were theoretically predicted. All the near-field SERS bands and corresponding band assignments were tabulated and are provided below. To this extent, near-field SERS mapping for individual bands of R6G were extracted and elaborated. At “SP3”, some of the bands of near-field SERS spectrum of R6G were found enhanced. However, at “SP1, SP2” and “SP5”, the band intensities of the near field SERS spectrum of R6G were found to be almost negligible compared to those obtained at “SP4” and “SP3”. The site “SP4” represents the strongest site of near-field SERS enhancement, which corresponds to the interstitial of the constituent nanoparticles “3” and “4” as shown in FIG. 5A. The incident laser had a polarization angle of 41° that was closely matched with the interparticle axis of constituent nanoparticles “3” and “4”. Therefore, the interstitial between nanoparticles “3” and “4” was in fact the hotspot; a site of strongly localized plasmon coupling. On the other hand, the site “SP3” was found closely located to the site “SP4” and thus strong near-field SERS of R6G was observed. A 3D hawks-eye view of the same near-field SERS mapping (1647 cm⁻¹, aromatic C—C stretching mode of R6G) is shown in FIG. 5C. A strongest SERS enhancement was noted at site “SP4” showing a highest peak, whereas the one next to the strongest enhancement was found at a shoulder. A contour plot of the near-field SERS intensity (1647 cm−1, aromatic C—C stretching mode of R6G) overlapped with near-field SERS mapping is shown in FIG. 5D. The contour plot reaffirms that the strongest near-field SERS enhancement was observed at site “SP4”.

As described, some of the bands of near-field SERS of R6G were found enhanced at specific sites, whereas at other sites, some bands were found weak. The spectrum of near-field SERS is mostly similar to the spectrum of far-field SERS, with the exception that the EM near-field is very intense in the near-field scenario due to the effect of the evanescent field. In addition to EM near-field, the orientation of the analyte also influences the intensity of a particular near-field SERS band. Some specific and selective modes of enhancement of R6G molecules were observed, which had never been reported even by single-molecule SERS detection, with an enhancement factor of up to 1015. The observed Raman shifts matched partially with the simulated values, considering the tip-enhancement effect. The SERS bands found in this experiment are summarized in Table 1, along with band assignments and comparisons with those reported experimentally and theoretically.

TABLE 1
SERS bands and corresponding band assignments of
R6G obtained under this investigation as well as
those obtained experimentally and theoretically.

| Mode of vibration | Experimental | Reference 1 | Reference 2 |
|---|---|---|---|
| | 1746 | | 1716 |
| C—C stretching in xanthene | 1647 | 1647 | 1652 |
| ring | | | |
| hybrid mode (phenyl ring with | 1622 | | 1615 |
| COOC2H5 | | | |
| | | 1596 | |
| C—C stretching in phenyl | 1572 | 1570 | 1577 |
| ring | | | |
| C—N stretching in NHC2H5 | 1458 | | 1458 |
| | | 1532 | 1419 |
| | 1422 | | |
| | | 1503 | |
| C—C stretching in xanthene | 1329 | 1359 | 1351 |
| ring | | | |
| | | 1308 | |
| | 1238 | 1269 | |
| C—H in-plane bending in | 1185 | 1185 | 1192 |
| xanthene ring | | | |
| | | 1120 | |
| | 1078 | 1084 | 1084 |
| | 887 | 919 | 895 |
| | 832 | | 819 |
| C—C op bend | 748 | 766 | 771 |
| | 649 | | 631 |
| | | 608 | |

Reference 1: See: Hayazawa, N., Inouye, Y., Sekkat, Z. and Kawata, S., 2002. Near-field Raman imaging of organic molecules by an apertureless metallic probe scanning optical microscope; The Journal of chemical physics, 117(3), pp. 1296-1301, incorporated as a reference in its entirety.
Reference 2: Watanabe, H., Hayazawa, N., Inouye, Y. and Kawata, S., 2005. DFT vibrational calculations of rhodamine 6G adsorbed on silver: analysis of tip-enhanced Raman spectroscopy. The Journal of Physical Chemistry B, 109(11), pp. 5012-5020, incorporated as a reference in its entirety.

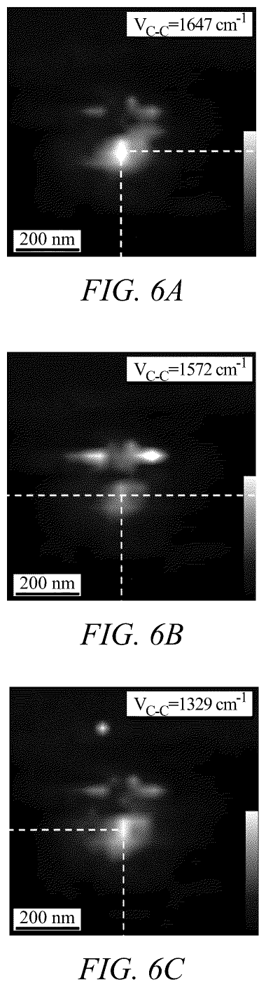

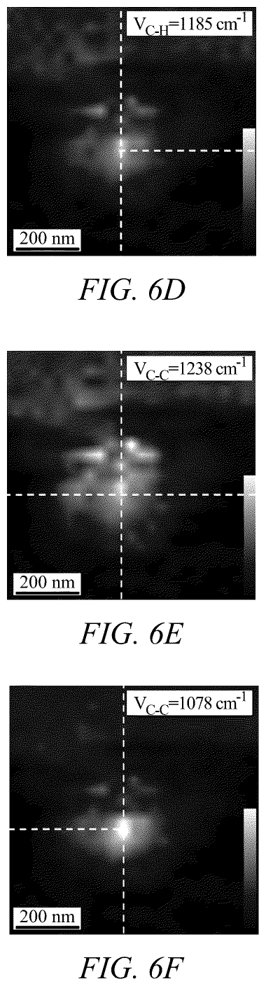

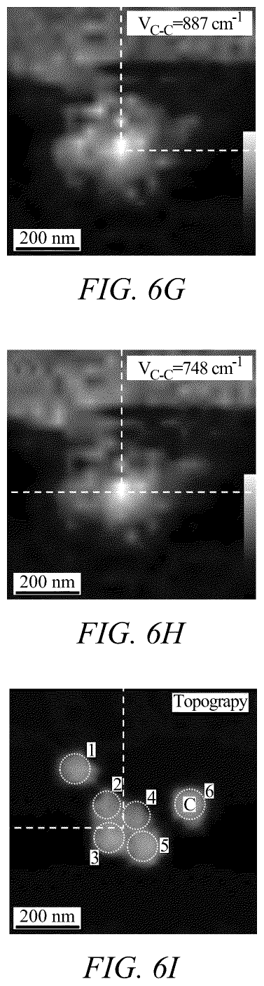

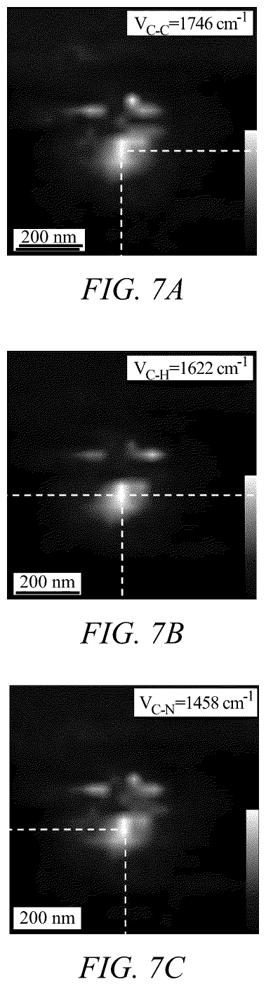

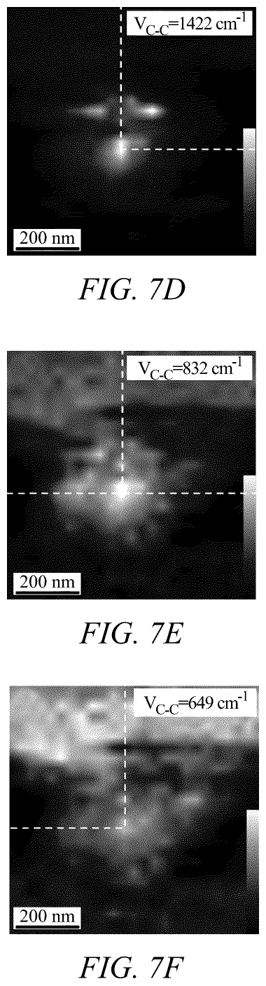

It was reported in reference 1 that many selective modes of enhancement of R6G molecules were possible by tip-enhanced experimental set-up in the near field, such as near-field SERS by a-NSOM used in the disclosure. As shown above in FIG. 5B, the near-field SERS spectrum of R6G at the right hotspot “SP4” indicated possible bands located at 1746, 1647, 1622, 1572, 1458, 1422, 1329, 1238, 1185, 1078, 887, 832, 748, and 649 cm⁻¹ wavenumbers. Some of these SERS bands, such as bands at 1647, 1572, 1329, 1238, 1185, 1078, 887, and 748 cm⁻¹ wavenumbers, coincided well with those reported experimentally by the reference 1, although a bit shifted peaks are well-acknowledged in near-field SERS measurements. On the other hand, all the SERS bands observed under this investigation were supported by the density functional theory reported theoretically by the reference 2. Further to clarify the correlation between near-field SERS-active sites and nanoscale topography, a near-field SERS mapping was extracted for the abovementioned SERS bands of R6G, as shown in FIG. 6A-FIG. 6I and FIG. 7A-FIG. 7F.

FIG. 6A-FIG. 6I show near-field SERS mappings for the selective SERS bands of R6G observed during experimentation, which coincided well with the experimentally reported works. FIG. 6A-FIG. 6H represent near-field SERS mappings of R6G extracted for the bands located at 1647, 1572, 1329, 1238, 1185, 1078, 887, and 748 cm⁻¹ wavenumbers, respectively. FIG. 6I displays a shear-force topography of the same area captured simultaneously during near-field SERS measurements. The dashed squares, as shown in FIG. 6A-FIG. 6I leads to sites of the strongest near-field SERS intensity for each individual band. It is noteworthy that the vertices of the four squares (when FIG. 6A-FIG. 6I are arranged together) are pointing at the strongest sites, and all the strongest sites matched well to the interstitial “SP4” in the topography, as shown in FIG. 6I. As stated in the preceding text, interstitial “SP4” facilitated localization of the strong EM near-field and thus a strong enhancement in SERS characteristics.

It was reported by many groups, including reference 1, that in near-field SERS measurements, it was possible to have some selective SERS bands that are not enhanced enough in far-field SERS measurements. In fact, reference 1 reported some near-field SERS bands of R6G that were not even supported by DFT calculations accomplished by many groups, including reference 1. At hotspots, near-field effects, such as evanescent fields, have a strong influence on near-field SERS measurements. In current disclosure, some selective near-field SERS bands of R6G were observed that were not reported experimentally. However, these bands of R6G were well supported by DFT calculations done by reference. FIG. 7A-FIG. 7I display near-field SERS mappings of these bands to demonstrate the correlation amongst strong SERS-active sites observed in individual SERS bands. FIG. 6A-FIG. 6F show near-field SERS mappings of R6G extracted for the bands located at 1746, 1622, 1458, 1422, 832, and 649 cm⁻¹ wavenumbers, respectively. The dashed squares, as mentioned in FIG. 7A-FIG. 7I, leads to sites of the strongest near-field SERS intensity for each individual band. It can be noted that the vertices of the four squares point at the strongest sites, and all the strongest sites match well to the interstitial “SP4” in the topography, as elaborated in the preceding section. It is well-known that the inherent nano-geometry of a particular interstitial determines how intense the localized EM near-field will be available to enhance the analyte laying at the same hotspot.

EM near-field distributions are described herein. There are two main mechanisms for SERS: the electromagnetic (EM) mechanism and the chemical (CM) mechanism. The EM mechanism is based on the enhancement of the electromagnetic field induced by the localized surface plasmons of the metal nanostructures, which enhances the Raman signal of the analyte by several orders of magnitude. The CM mechanism, on the other hand, is based on the chemical interaction between the metal surface and the analyte, which results in a new Raman-active molecule or complex that exhibits a stronger Raman signal. The EM mechanism is the most prominent mechanism in SERS, and it is based on the excitation of surface plasmons, which are collective oscillations of the electrons in the metal nanostructures. When the plasmons are excited by the incident light, they generate a strong electromagnetic field that is highly localized around the metal nanostructures, leading to a substantial enhancement of the Raman signal of the analyte molecules in the near-field region. The CM mechanism, while less well-understood and less common, is often used to selectively enhance specific Raman modes of the analyte, overcoming some of the challenges associated with obtaining reproducible SERS measurements. A two-fold EM enhancement mechanism may be used for EM enhancement in the SERS process. In the first phase, as defined in equation (1), EM near-field contributes to improving analyte Raman scattering, whereas the second process begins by improving scattered Raman light from adsorbed analytes. Therefore, the enhancement factor, M (including the first factor, M1, and the second factor, M2) for SERS is denoted by

$$M = {\vert \frac{{E}_{L} ( {λ}_{I} )}{{E}_{I} ( {λ}_{I} )} \vert}^{2} \times {\vert \frac{{E}_{L} ( {λ}_{I} \pm {λ}_{R} )}{{E}_{I} ( {λ}_{I} \pm {λ}_{R} )} \vert}^{2} = {M}_{1} ( {λ}_{I} ) \times {M}_{2} ( {λ}_{I} \pm {λ}_{R} ) , \qquad ( 1 )$$

where E_{I}, E_{L}, λ_{I}, +λ_{R} and −λ_{R} Rare the incident electric fields, local electric fields, excitation wavelength, wavelengths of the anti-Stokes and Stokes Raman shifts respectively.

Near-field SERS spectroscopy of a well-defined gold nanoaggregate adsorbed with the Raman-active dye R6G was performed. An a-NSOM setup facilitated recording spectrally- and spatially-resolved SERS measurements at the very same position without disturbing the specimen. A direct observation of a SERS-active hotspot was realized through near-field SERS measurements. A well defined correlation between nanometric geometry and optical image was obtained through simultaneous measurements of shear-force topography and near-field SERS. The interstitial positioned at the center of the nanoaggregate provided the most intense Raman scattering, implying the possibility of a SERS hotspot therein. SERS bands of the spectrum of the Raman-active dye Rhodamine 6G recorded at the same hotspot coincided well with those reported so far. An FDTD model very similar to that under investigation was developed, and EM near-field distributions were extracted. A strong and localized EM near-field distribution at the interstitial positioned at the center of the tetramer was obtained, correlating well with the SERS hotspot observed in near-field SERS measurements.

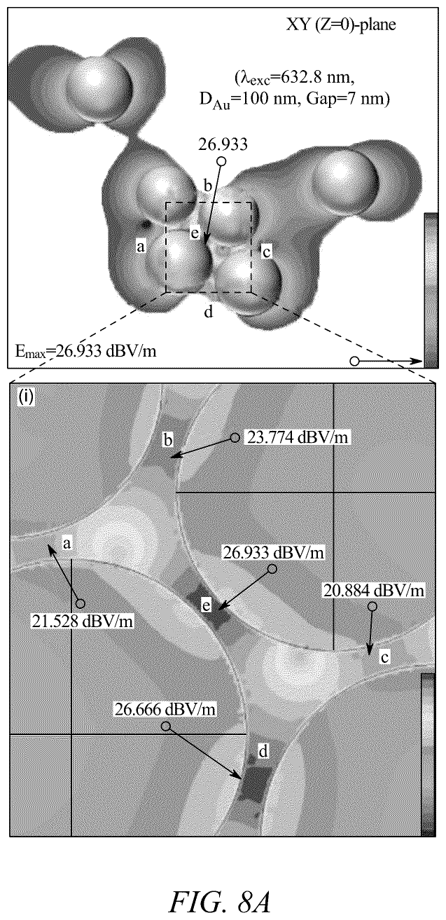

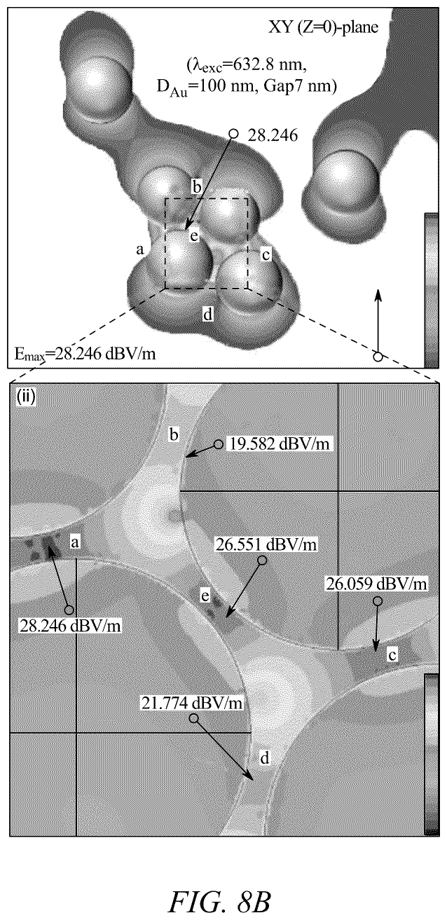

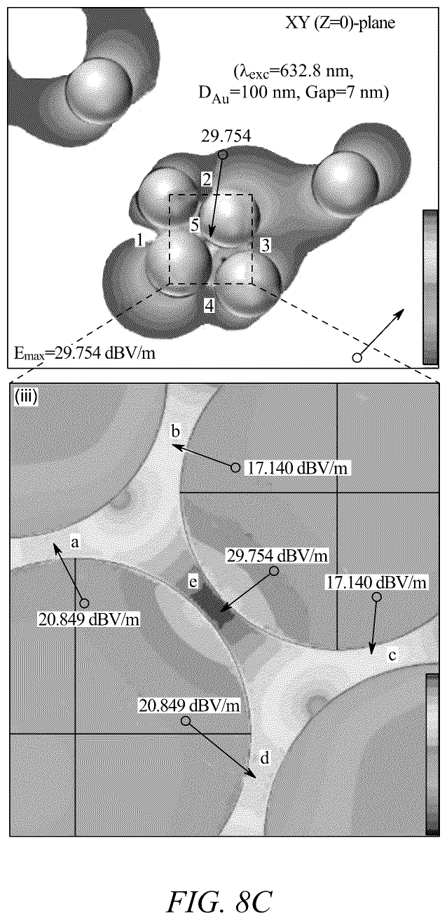

Since EM near-field distribution is the main ingredient in SERS enhancement, EM near-field distributions were extracted in this context for typical models as shown in FIG. 6. The model was designed and simulated for s-, p- and 45° of incident polarizations. Excitation of 632.8 nm that was normal to the geometry was used according to the experimental conditions. Five interstitials as marked by “a”, “b”, “c”, “d” and “e” were particularly highlighted and interestingly, these five interstitials corresponded to the interstitials as elaborated in FIG. 4A. The interstitials “a” and “c” represents the interstitials along the axis of 1000 (between nanoparticles “2” and “3” and nanoparticles “4” and “5” respectively) whereas the interstitials “b” and “d” corresponds to those along the axis of 1600 (between nanoparticles “2” and “4” and nanoparticles “3” and “5” respectively). And the interstitial “e” corresponds to the interstitial along the axis of 400 (between nanoparticles “2” and “4”). FIG. 8A represents EM near-field distributions at XY (Z=0) plane for a typical model geometry excited with incident excitation of s-polarization. Maximum EM near-field intensity of 26.933 dBV/m was found to be confined at the interstitial “e”, although the interparticle axis was out of plane to the incident excitation. A zoom-in view covering all the five interstitials was shown in inset (i) of FIG. 8A. It can be noted that the interstitials “e” and “d” were having almost similar intensities of EM near-field distribution (26.933 and 26.666 dBV/m respectively), although the interparticle axes were along ˜40⁰ and ˜160⁰ respectively. Maximum intensities of EM near-field distribution available at interstitials “a”, “b” and “c” were estimated to be 21.528, 23.774 and 20.884 dBV/m respectively as shown in inset (i) of FIG. 8A. The interparticle axes of interstitials “b” and “d” were found to be closer to s-polarized incident excitation compared to those of interstitials “a” and “c”. It has been known that interstitial of interparticle axis parallel to incident polarization facilitates strong EM near-field localization. Due to this aspect, strong EM near-field distribution is observed at interstitial “d”. However, the interstitial “e” was having the strongest EM near-field distribution at s-polarization of incident excitation, although the corresponding interparticle axis was far away, ˜40° with reference to s-polarization. This indicated that the nano-geometry of the interstitial is dominant over the influence of incident polarization in localizing EM near-field distribution. Under this circumstance, the interstitial having strong EM near-field distribution will provide strong enhancement in SERS measurements as observed herewith and demonstrated above.

In the case of p-polarization of incident excitation, it was expected that the interstitial of the interparticle axis parallel to incident excitation would support a strong EM near-field distribution. FIG. 8B shows EM near-field distributions along the XY (Z=0) plane of the same model excited with the p-polarization of incident excitation. As expected, the interstitials of the interparticle axis closely parallel to incident polarization were found to have strong EM near-field distributions. As stated earlier, the interparticle axes of interstitials “a” and “c” were found to be 100°, which is nearly parallel to the p-polarization of incident excitation. Therefore, the maximum intensities of EM near-field distributions at interstitials “a” and “c” were estimated to be 28.246 and 26.059 dBV/m, respectively, as shown in inset (i) of FIG. 8B. Inset (i) of FIG. 8B represents a zoom-in view of the selected area as marked by the white dashed square in FIG. 8B. As expected, the interstitials “b” and “d” had lower intensities of EM near-field distributions (Emax=19.582 and 21.774 dBV/m, respectively) due to out of plane interparticle axes with reference to incident excitation. The interstitial “e” had a reasonably high intensity (Emax=26.551 dBV/m) of EM near-field distribution similar to that occurring at s-polarization, although the corresponding interparticle axis was estimated to be ˜40°. It was noteworthy that the interstitial “e” was more favorable in localizing EM near-field distribution regardless of incident excitation polarizations. In the case of oblique polarization (45°) of incident excitation, the interstitial “e” showed the strongest EM near-field distribution, as shown in FIG. 8C. FIG. 8C displays EM near-field distributions along XY (Z=0) plane of the same model excited with incident excitation of oblique) (45°-polarization. As expected, the interstitial “e” had the strongest EM near-field distribution (Emax=29.754 dBV/m) due to its interparticle axis being closely parallel to the incident excitation polarization, as shown in inset (i) of FIG. 8C. Inset (i) of FIG. 8C depicts a zoom-in view of the selected area as marked by the white dashed square in FIG. 8C. It is to be noted that the intensities of interstitials “b” and “c” (Emax=17.140 dBV/m) and those of interstitials “a” and “d” (Emax=20.849 dBV/m) were found lower due to out-of-plane interparticle axes with reference to incident excitation of oblique) (45°-polarization.

The first embodiment is illustrated with respect to FIG. 1A-FIG. 8C. A method for functionalizing substrates for use in near field surface-enhanced Raman scattering (SERS) spectroscopy is described. The method includes coating each functionalized glass substrate of a set of glass substrates functionalized with trimethoxy-[3-(methylamino)propyl] silane with a colloidal solution of gold nanoparticles suspended in water. The trimethoxy-[3-(methylamino)propyl] silane immobilizes the gold nanoparticles on each functionalized glass substrate. The method also includes applying a Raman-active dye to the glass substrate having immobilized gold nanoparticles. In addition, the method includes performing near field SERS spectroscopy of each functionalized glass substrate 102 coated with dyed immobilized gold nanoparticles and identifying, from the near-field SERS spectroscopy, for each functionalized glass substrate 102 coated with dyed immobilized gold nanoparticles, hotspots which produce high intensity scattering from the dyed immobilized gold nanoparticles. Each hotspot is located at an interstitial position between two adjacent dyed immobilized gold nanoparticles. The method includes identifying for each functionalized glass substrate 102 coated with dyed immobilized gold nanoparticles, a direction of an interparticle axis between the two adjacent dyed immobilized gold nanoparticles and an electromagnetic near field intensity of the high intensity scattering along the direction of the interparticle axis for each hotspot. The method also includes mapping for each functionalized glass substrate 102 coated with dyed immobilized gold nanoparticles, by a computing device 108, a location of each interstitial position of each hotspot, the direction of the interparticle axis of the respective hotspot, and the electromagnetic near field intensity of the respective hotspot. The method further includes assigning a substrate identification number to each glass substrate 102 coated with dyed immobilized gold nanoparticles, and storing, in a database 120, the location of each interstitial position of each hotspot, the direction of the interparticle axis of the respective hotspot, and the electromagnetic near field intensity of the respective hotspot with the substrate identification number.

In one aspect, the method includes spin coating the Raman-active dye 104 onto each functionalized glass substrate 102 coated with the immobilized gold nanoparticles, wherein spin coating distributes the Raman-active dye 104 across each functionalized glass substrate 102 coated with the immobilized gold nanoparticles. The gold nanoparticles each have a diameter in a range of 96.0 nm to 104.0 nm.

In one aspect, the method includes selecting a functionalized glass substrate 102 coated with dyed immobilized gold nanoparticles, retrieving, from the database 120, the location of each interstitial position of each hotspot, the direction of the interparticle axis of the respective hotspot, and the electromagnetic near field intensity of the respective hotspot based on the substrate identification number of functionalized glass substrate 102 coated with dyed immobilized gold nanoparticles, coating a target analyte on the glass substrate 102 having the substrate identification number, performing, with an aperture near-field scanning optical microscope (a-NSOM 106), for each hotspot, near field SERS spectroscopy using an incident beam having a p-polarization parallel to the direction of the interparticle axis of the respective hotspot, recording the electromagnetic near field intensity at each hotspot, and identifying the target analyte by matching, by the computing device 108, the electromagnetic field intensity of each hotspot to a known electromagnetic near field intensity of the target analyte.

In an aspect, the method includes selecting the Raman-active dye 104 based on the target analyte.

Performing near field SERS spectroscopy with the a-NSOM 106 includes equipping the a-NSOM 106 with a tapered probe and emitting the incident beam having a p-polarization from a tip of the tapered probe.

In one aspect, the method includes depositing a gold coating on the tip of the tapered probe.

In one aspect, the method includes identifying the direction of the interparticle axis between the two adjacent dyed immobilized gold nanoparticles by performing sheer force measurements simultaneously with the near-field SERS measurements by using the a-NSOM 106 with the tapered probe and generating a contour map configured to show the interparticle axes of each hotspot.

The second embodiment is illustrated with respect to FIG. 1-FIG. 8C. A method of identifying a target analyte is described. The method includes obtaining a functionalized glass substrate 102 coated with dyed immobilized gold nanoparticles and coating the functionalized glass substrate 102 coated with dyed immobilized gold nanoparticles with a target analyte. The functionalized glass substrate 102 coated with dyed immobilized gold nanoparticles has known positions of high intensity scattering from interparticle axes between adjacent dyed immobilized gold nanoparticles. In addition, the method includes performing near field SERS spectroscopy at the known positions by directing a laser beam having a p-polarization along a direction parallel to a direction of the interparticle axis of each known position, receiving, by a computing device 108, SERS spectra for each of the known positions, comparing the SERS spectra for each of the known positions to a database 120 record of known SERS spectra of molecules and identifying molecules in the target analyte based on matching the SERS spectra to the database record of known SERS spectra of molecules. In an aspect, the method includes performing near field SERS spectroscopy by directing a tip of a tapered probe of an aperture near-field scanning optical microscope (a-NSOM) in the direction of the interparticle axis of each known position. The tip of the tapered probe is coated with gold.

The glass substrate 102 having immobilized gold nanoparticles is coated with a dye by selecting a dye based on the target analyte, obtaining the selected dye, applying a drop of the selected dye to the glass substrate 102 having immobilized gold nanoparticles, inserting the functionalized glass substrate 102 coated with the immobilized gold nanoparticles into a spin coating machine, and spin coating the dye onto each functionalized glass substrate 102 coated with the immobilized gold nanoparticles, The spin coating distributes the dye across each functionalized glass substrate 102 coated with the immobilized gold nanoparticles.

The dye is a Raman-active dye 104. The Raman-active dye 104 is Rhodamine 6G.

The gold nanoparticles each have a diameter in a range of 96.0 nm to 104.0 nm.

Each functionalized glass substrate 102 coated with dyed immobilized gold nanoparticles is tagged with a substrate identification number.

In one aspect, the method includes identifying hotspots on each functionalized glass substrate 102 coated with dyed immobilized gold nanoparticles by detecting positions of high intensity scattering, identifying interstitials of adjacent dyed immobilized gold nanoparticles at each hotspot, identifying a direction of an interparticle axis of each interstitial of each respective hotspot, storing, in the database 120 record, the location of each interstitial position of each hotspot, the direction of the interparticle axis of each respective hotspot, and an electromagnetic near field intensity of the respective hotspot with the substrate identification number.

Identifying the direction of the interparticle axis between the two adjacent dyed immobilized gold nanoparticles at each hotspot includes performing sheer force measurements simultaneously during the near-field SERS measurements using the a-NSOM 106 with the tapered probe, generating a contour map based on the sheer force measurements, determining the hotspots from the positions of high intensity scattering observed from the near-field SERS measurements, determining the interparticle axes of each hotspot from the contour map, and determining a direction of the interparticle axis of each hotspot by observing the strength of the high intensity scattering along each interparticle axis.

The third embodiment is illustrated with respect to FIG. 1A-FIG. 8C. A system 100 for functionalizing glass substrates 102 for use in detecting target molecules with near field surface-enhanced Raman scattering (SERS) spectroscopy is described. The system 100 includes a glass substrate 102 coated with immobilized gold nanoparticles. Each glass substrate 102 has an identification number. The system 100 includes a coating of Raman-active dye 104 configured to adhere to the molecule in the target analyte. The coating of Raman-active dye 104 is applied by spin coating. In addition, the system 100 includes an aperture near-field scanning optical microscope (a-NSOM 106) configured to perform near field SERS spectroscopy of each functionalized glass substrate 102 coated with the dyed immobilized gold nanoparticles and a computing device connected to the a-NSOM 106. The computing device 108 includes electric circuitry, a memory configured to store program instructions and at least on processor configured to execute the program instructions to identify, for each functionalized glass substrate 102 coated with the dyed immobilized gold nanoparticles, positions of hotspots which produce high intensity scattering from the dyed immobilized gold nanoparticles. Each hotspot is located at an interstitial position between two adjacent dyed immobilized gold nanoparticles. The a-NSOM 106 is further configured to perform sheer force measurements simultaneously with the near-field SERS spectroscopy and generate a contour map configured to show the interparticle axes of each hotspot. The a-NSOM 106 is further configured to measure an electromagnetic near field intensity along the interparticle axes of each hotspot. The computing device 108 is further configured to identify a direction of the interparticle axis between the two adjacent dyed immobilized gold nanoparticles of each hotspot based on the contour map. The system 100 also includes a database 120 connected to the computing device 108. The database 120 is configured to store the identification number of each glass substrate 102, the position of each hotspot, the direction of the interparticle axis between the two adjacent dyed immobilized gold nanoparticles of each hotspot, the electromagnetic near field intensity along the interparticle axes of each hotspot and the contour map.

In one aspect, the system 100 includes a target analyte which includes an unknown molecule. The unknown solution is applied to an outer surface of a selected functionalized glass substrate 102 coated with the dyed immobilized gold nanoparticles. A computing device 108 is configured to retrieve the positions of each hotspot and the directions of the interparticle axes from the database 120 based on an identification number of the selected functionalized glass substrate 102. The a-NSOM 106 is configured to perform near field SERS spectroscopy at the hotspots by directing a laser beam having a p-polarization along a direction parallel to a direction of the interparticle axis of each hotspot and recording the electromagnetic near field intensity along the interparticle axis of each hotspot. The computing device 108 is configured to receive the electromagnetic near field intensity for each of the hotspots, compare the electromagnetic near field intensity for each of the hotspots to a database 120 record of known SERS spectra of molecules, and identify molecules in the target analyte based on matching the electromagnetic near field intensity for each of the hotspots to the database 120 record of known SERS spectra of molecules.

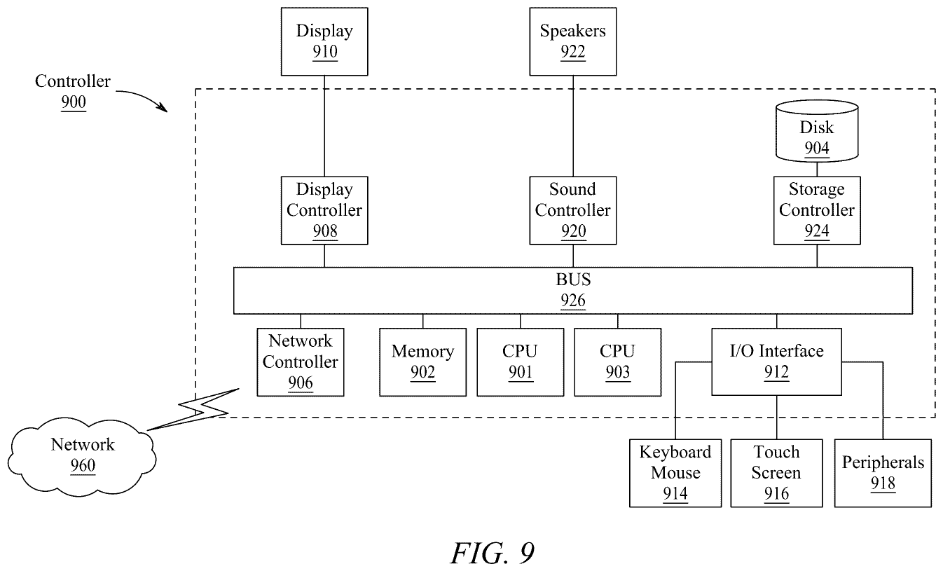

Next, further details of the hardware description of the computing environment according to exemplary embodiments is described with reference to FIG. 1. In FIG. 9, a controller 900 is described is representative of the system 100 of FIG. 1 in which the controller is the computing device 108 which includes a CPU 901 which performs the processes described above/below. The process data and instructions may be stored in memory 902. These processes and instructions may also be stored on a storage medium disk 904 such as a hard drive (HDD) or portable storage medium or may be stored remotely.

Further, the claims are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the computing device communicates, such as a server or computer.

Further, the claims may be provided as a utility application, background daemon, or component of an operating system, or combination thereof, executing in conjunction with CPU 901, 903 and an operating system such as Microsoft Windows 9, Microsoft Windows 12, Microsoft Windows 11, UNIX, Solaris, LINUX, Apple MAC-OS and other systems known to those skilled in the art.

The hardware elements in order to achieve the computing device may be realized by various circuitry elements, known to those skilled in the art. For example, CPU 901 or CPU 903 may be a Xenon or Core processor from Intel of America or an Opteron processor from AMD of America, or may be other processor types that would be recognized by one of ordinary skill in the art. Alternatively, the CPU 901, 903 may be implemented on an FPGA, ASIC, PLD or using discrete logic circuits, as one of ordinary skill in the art would recognize. Further, CPU 901, 903 may be implemented as multiple processors cooperatively working in parallel to perform the instructions of the inventive processes described above.

The computing device in FIG. 9 also includes a network controller 906, such as an Intel Ethernet PRO network interface card from Intel Corporation of America, for interfacing with network 960. As can be appreciated, the network 960 can be a public network, such as the Internet, or a private network such as an LAN or WAN network, or any combination thereof and can also include PSTN or ISDN sub-networks. The network 960 can also be wired, such as an Ethernet network, or can be wireless such as a cellular network including EDGE, 3G, 4G and 5G wireless cellular systems. The wireless network can also be WiFi, Bluetooth, or any other wireless form of communication that is known.

The computing device further includes a display controller 908, such as a NVIDIA Geforce GTX or Quadro graphics adaptor from NVIDIA Corporation of America for interfacing with display 910, such as a Hewlett Packard HPL2445w LCD monitor. A general purpose I/O interface 912 interfaces with a keyboard and/or mouse 914 as well as a touch screen panel 916 on or separate from display 910. General purpose I/O interface also connects to a variety of peripherals 918 including printers and scanners, such as an OfficeJet or DeskJet from Hewlett Packard. A sound controller 920 is also provided in the computing device such as Sound Blaster X-Fi Titanium from Creative, to interface with speakers/microphone 922 thereby providing sounds and/or music.

The general purpose storage controller 924 connects the storage medium disk 904 with communication bus 926, which may be an ISA, EISA, VESA, PCI, or similar, for interconnecting all of the components of the computing device. A description of the general features and functionality of the display 910, keyboard and/or mouse 914, as well as the display controller 908, storage controller 924, network controller 906, sound controller 920, and general purpose I/O interface 912 is omitted herein for brevity as these features are known.

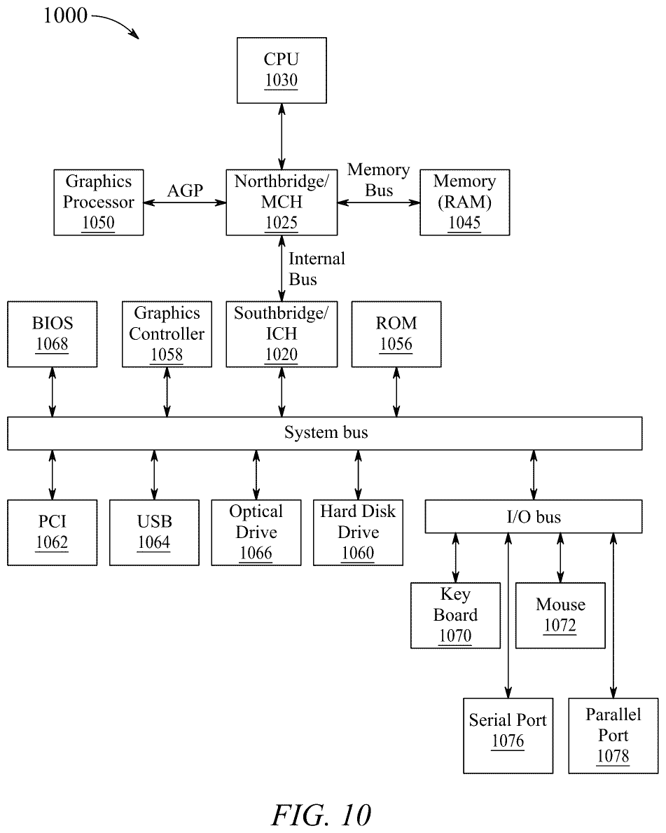

The exemplary circuit elements described in the context of the present disclosure may be replaced with other elements and structured differently than the examples provided herein. Moreover, circuitry configured to perform features described herein may be implemented in multiple circuit units (e.g., chips), or the features may be combined in circuitry on a single chipset, as shown on FIG. 10. FIG. 10 shows a schematic diagram of a data processing system, according to certain embodiments, for performing the functions of the exemplary embodiments. The data processing system is an example of a computer in which code or instructions implementing the processes of the illustrative embodiments may be located. In FIG. 10, data processing system 1000 employs a hub architecture including a north bridge and memory controller hub (NB/MCH) 1025 and a south bridge and input/output (I/O) controller hub (SB/ICH) 1020. The central processing unit (CPU) 1030 is connected to NB/MCH 1025. The NB/MCH 1025 also connects to the memory 1045 via a memory bus, and connects to the graphics processor 1050 via an accelerated graphics port (AGP). The NB/MCH 1025 also connects to the SB/ICH 1020 via an internal bus (e.g., a unified media interface or a direct media interface). The CPU Processing unit 1030 may contain one or more processors and even may be implemented using one or more heterogeneous processor systems.

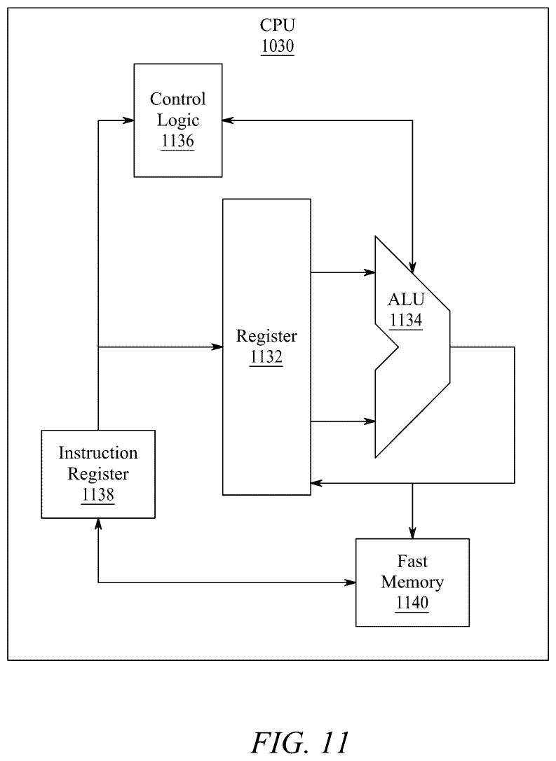

For example, FIG. 11 shows one implementation of CPU 1030. In one implementation, the instruction register 1138 retrieves instructions from the fast memory 1140. At least part of these instructions are fetched from the instruction register 1138 by the control logic 1136 and interpreted according to the instruction set architecture of the CPU 1030. Part of the instructions can also be directed to the register 1132. In one implementation the instructions are decoded according to a hardwired method, and in another implementation the instructions are decoded according a microprogram that translates instructions into sets of CPU configuration signals that are applied sequentially over multiple clock pulses. After fetching and decoding the instructions, the instructions are executed using the arithmetic logic unit (ALU) 1134 that loads values from the register 1132 and performs logical and mathematical operations on the loaded values according to the instructions. The results from these operations can be feedback into the register and/or stored in the fast memory 1140. According to certain implementations, the instruction set architecture of the CPU 1030 can use a reduced instruction set architecture, a complex instruction set architecture, a vector processor architecture, a very large instruction word architecture. Furthermore, the CPU 1030 can be based on the Von Neuman model or the Harvard model. The CPU 1030 can be a digital signal processor, an FPGA, an ASIC, a PLA, a PLD, or a CPLD. Further, the CPU 1030 can be an x86 processor by Intel or by AMD; an ARM processor, a Power architecture processor by, e.g., IBM; a SPARC architecture processor by Sun Microsystems or by Oracle; or other known CPU architecture.

Referring again to FIG. 10, the data processing system 1000 can include that the SB/ICH 1020 is coupled through a system bus to an I/O Bus, a read only memory (ROM) 1056, universal serial bus (USB) port 1064, a flash binary input/output system (BIOS) 1068, and a graphics controller 1058. PCI/PCIe devices can also be coupled to SB/ICH 1088 through a PCI bus 1062.

The PCI devices may include, for example, Ethernet adapters, add-in cards, and PC cards for notebook computers. The Hard disk drive 1060 and CD-ROM 1066 can use, for example, an integrated drive electronics (IDE) or serial advanced technology attachment (SATA) interface. In one implementation the I/O bus can include a super I/O (SIO) device.

Further, the hard disk drive (HDD) 1060 and optical drive 1066 can also be coupled to the SB/ICH 1020 through a system bus. In one implementation, a keyboard 1070, a mouse 1072, a parallel port 1078, and a serial port 1076 can be connected to the system bus through the I/O bus. Other peripherals and devices that can be connected to the SB/ICH 1020 using a mass storage controller such as SATA or PATA, an Ethernet port, an ISA bus, a LPC bridge, SMBus, a DMA controller, and an Audio Codec.

Moreover, the present disclosure is not limited to the specific circuit elements described herein, nor is the present disclosure limited to the specific sizing and classification of these elements. For example, the skilled artisan will appreciate that the circuitry described herein may be adapted based on changes on battery sizing and chemistry, or based on the requirements of the intended back-up load to be powered.

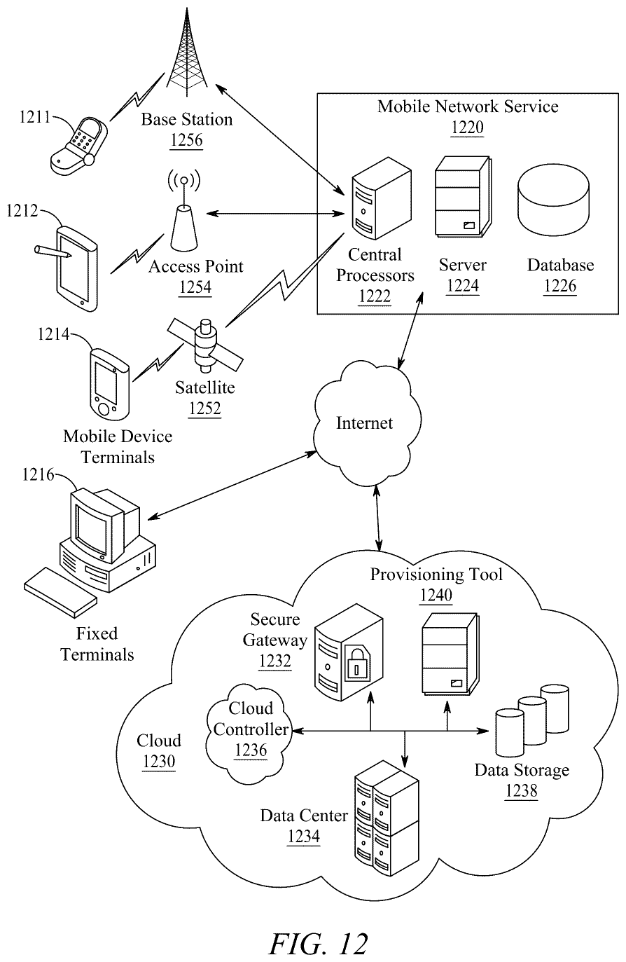

The functions and features described herein may also be executed by various distributed components of a system. For example, one or more processors may execute these system functions, wherein the processors are distributed across multiple components communicating in a network. The distributed components may include one or more client and server machines, which may share processing, as shown by FIG. 12, in addition to various human interface and communication devices (e.g., display monitors, smart phones, tablets, personal digital assistants (PDAs)). The network may be a private network, such as a LAN or WAN, or may be a public network, such as the Internet. Input to the system may be received via direct user input and received remotely either in real-time or as a batch process. Additionally, some implementations may be performed on modules or hardware not identical to those described. Accordingly, other implementations are within the scope that may be claimed.

The above-described hardware description is a non-limiting example of corresponding structure for performing the functionality described herein.

Numerous modifications and variations of the present disclosure are possible in light of the above teachings. It is therefore to be understood that within the scope of the appended claims, the invention may be practiced otherwise than as specifically described herein.

1. A method for functionalizing substrates for use in near field surface-enhanced Raman scattering (SERS) spectroscopy, comprising:
coating each functionalized glass substrate of a set of glass substrates functionalized with trimethoxy-[3-(methylamino)propyl] silane with a colloidal solution of gold nanoparticles suspended in water, wherein the trimethoxy-[3-(methylamino)propyl] silane immobilizes the gold nanoparticles on each functionalized glass substrate;
applying a Raman-active dye to the glass substrate having immobilized gold nanoparticles;
performing near field SERS spectroscopy of each functionalized glass substrate coated with dyed immobilized gold nanoparticles; and
identifying, from the near field SERS spectroscopy, for each functionalized glass substrate coated with dyed immobilized gold nanoparticles, hotspots which produce high intensity scattering from the dyed immobilized gold nanoparticles, wherein each hotspot is located at an interstitial position between two adjacent dyed immobilized gold nanoparticles;
identifying for each functionalized glass substrate coated with dyed immobilized gold nanoparticles, a direction of an interparticle axis between the two adjacent dyed immobilized gold nanoparticles and an electromagnetic near field intensity of the high intensity scattering along the direction of the interparticle axis for each hotspot;
mapping for each functionalized glass substrate coated with dyed immobilized gold nanoparticles, by a computing device, a location of each interstitial position of each hotspot, the direction of the interparticle axis of the respective hotspot, and the electromagnetic near field intensity of the respective hotspot;
assigning a substrate identification number to each glass substrate coated with dyed immobilized gold nanoparticles; and
storing, in a database, the location of each interstitial position of each hotspot, the direction of the interparticle axis of the respective hotspot, and the electromagnetic near field intensity of the respective hotspot with the substrate identification number.

2. The method of claim 1, further comprising:
spin coating the Raman-active dye onto each functionalized glass substrate coated with the immobilized gold nanoparticles, wherein spin coating distributes the Raman-active dye across each functionalized glass substrate coated with the immobilized gold nanoparticles.

3. The method of claim 1, wherein the gold nanoparticles each have a diameter in a range of 96.0 nm to 104.0 nm.

4. The method of claim 1, further comprising:
selecting a functionalized glass substrate coated with dyed immobilized gold nanoparticles;
retrieving, from the database, the location of each interstitial position of each hotspot, the direction of the interparticle axis of the respective hotspot, and the electromagnetic near field intensity of the respective hotspot based on the substrate identification number of functionalized glass substrate coated with dyed immobilized gold nanoparticles;
coating a target analyte on the glass substrate having the substrate identification number;
performing, with an aperture near-field scanning optical microscope (a-NSOM), for each hotspot, near field SERS spectroscopy using an incident beam having a p-polarization parallel to the direction of the interparticle axis of the respective hotspot;
recording the electromagnetic near field intensity at each hotspot; and
identifying the target analyte by matching, by the computing device, the electromagnetic field intensity of each hotspot to a known electromagnetic near field intensity of the target analyte.

5. The method of claim 4, further comprising:
selecting the Raman-active dye based on the target analyte.

6. The method of claim 1, wherein performing near field SERS spectroscopy with the a-NSOM includes equipping the a-NSOM with a tapered probe and emitting the incident beam having a p-polarization from a tip of the tapered probe.

7. The method of claim 6, further comprising depositing a gold coating on the tip of the tapered probe.

8. The method of claim 6, further comprising:
identifying the direction of the interparticle axis between the two adjacent dyed immobilized gold nanoparticles by performing sheer force measurements simultaneously with the near-field SERS measurements by using the a-NSOM with the tapered probe and generating a contour map configured to show the interparticle axes of each hotspot.

9. A method of identifying a target analyte, comprising:
obtaining a functionalized glass substrate coated with dyed immobilized gold nanoparticles;
coating the functionalized glass substrate coated with dyed immobilized gold nanoparticles with a target analyte, wherein the functionalized glass substrate coated with dyed immobilized gold nanoparticles has known positions of high intensity scattering from interparticle axes between adjacent dyed immobilized gold nanoparticles;
performing near field SERS spectroscopy at the known positions by directing a laser beam having a p-polarization along a direction parallel to a direction of the interparticle axis of each known position;
receiving, by a computing device, SERS spectra for each of the known positions;
comparing the SERS spectra for each of the known positions to a database record of known SERS spectra of molecules; and
identifying molecules in the target analyte based on matching the SERS spectra to the database record of known SERS spectra of molecules.

10. The method of claim 9, performing the near field SERS spectroscopy by directing a tip of a tapered probe of an aperture near-field scanning optical microscope (a-NSOM) in the direction of the interparticle axis of each known position.

11. The method of claim 10, wherein the tip of the tapered probe is coated with gold.

12. The method of claim 9, wherein the glass substrate having immobilized gold nanoparticles is coated with a dye by the steps of:
selecting a dye based on the target analyte;
obtaining the selected dye;
applying a drop of the selected dye to the glass substrate having immobilized gold nanoparticles; and
inserting the functionalized glass substrate coated with the immobilized gold nanoparticles into a spin coating machine; and
spin coating the dye onto each functionalized glass substrate coated with the immobilized gold nanoparticles, wherein the spin coating distributes the dye across each functionalized glass substrate coated with the immobilized gold nanoparticles.

13. The method of claim 9, wherein the dye is a Raman-active dye.

14. The method of claim 13, wherein the Raman-active dye is Rhodamine 6G.

15. The method of claim 9, wherein the gold nanoparticles each have a diameter in a range of 96.0 nm to 104.0 nm.

16. The method of claim 9, wherein each functionalized glass substrate coated with dyed immobilized gold nanoparticles is tagged with a substrate identification number.

17. The method of claim 16, further comprising:
identifying hotspots on each functionalized glass substrate coated with dyed immobilized gold nanoparticles by detecting positions of high intensity scattering;
identifying interstitials of adjacent dyed immobilized gold nanoparticles at each hotspot;
identifying a direction of an interparticle axis of each interstitial of each respective hotspot;
storing, in the database record, the location of each interstitial position of each hotspot, the direction of the interparticle axis of each respective hotspot, and an electromagnetic near field intensity of the respective hotspot with the substrate identification number.

18. The method of claim 16, wherein identifying the direction of the interparticle axis between the two adjacent dyed immobilized gold nanoparticles at each hotspot comprises:
performing sheer force measurements simultaneously during the near-field SERS measurements using the a-NSOM with the tapered probe;
generating a contour map based on the sheer force measurements;
determining the hotspots from the positions of high intensity scattering observed from the near-field SERS measurements;
determining the interparticle axes of each hotspot from the contour map; and
determining a direction of the interparticle axis of each hotspot by observing the strength of the high intensity scattering along each interparticle axis.

19. A system for functionalizing glass substrates for use in detecting target molecules with near field surface-enhanced Raman scattering (SERS) spectroscopy, comprising:
a glass substrate coated with immobilized gold nanoparticles, wherein each glass substrate has an identification number;
a coating of Raman-active dye configured to adhere to the molecule in the target analyte, wherein the coating of Raman-active dye is applied by spin coating;
an aperture near-field scanning optical microscope (a-NSOM) configured to perform near field SERS spectroscopy of each functionalized glass substrate coated with the dyed immobilized gold nanoparticles; and
a computing device connected to the a-NSOM, wherein the computing device includes electrical circuitry, a memory configured to store program instructions and at least one processor configured to execute the program instructions to identify, for each functionalized glass substrate coated with the dyed immobilized gold nanoparticles, positions of hotspots which produce high intensity scattering from the dyed immobilized gold nanoparticles, wherein each hotspot is located at an interstitial position between two adjacent dyed immobilized gold nanoparticles,
wherein the a-NSOM is further configured to perform sheer force measurements simultaneously with the near-field SERS spectroscopy and generate a contour map configured to show the interparticle axes of each hotspot,
wherein the a-NSOM is further configured to measure an electromagnetic near field intensity along the interparticle axes of each hotspot,
wherein the computing device is further configured to identify a direction of the interparticle axis between the two adjacent dyed immobilized gold nanoparticles of each hotspot based on the contour map; and
a database connected to the computing device, wherein the database is configured to store the identification number of each glass substrate, the position of each hotspot, the direction of the interparticle axis between the two adjacent dyed immobilized gold nanoparticles of each hotspot, the electromagnetic near field intensity along the interparticle axes of each hotspot and the contour map.

20. The system of claim 19, further comprising:
a target analyte which includes an unknown molecule,
wherein the unknown solution is applied to an outer surface of a selected functionalized glass substrate coated with the dyed immobilized gold nanoparticles,
wherein the computing device is configured to retrieve the positions of each hotspot and the directions of the interparticle axes from the database based on an identification number of the selected functionalized glass substrate,
wherein the a-NSOM is configured to perform near field SERS spectroscopy at the hotspots by directing a laser beam having a p-polarization along a direction parallel to a direction of the interparticle axis of each hotspot and recording the electromagnetic near field intensity along the interparticle axis of each hotspot,
wherein the computing device is configured to receive the electromagnetic near field intensity for each of the hotspots, compare the electromagnetic near field intensity for each of the hotspots to a database record of known SERS spectra of molecules and identify molecules in the target analyte based on matching the electromagnetic near field intensity for each of the hotspots to the database record of known SERS spectra of molecules.
